(19) United States
(12) Patent Application Publication
(10) Pub. No.: US 2026/0130908 A1
(43) Pub. Date: May 14, 2026

(54) METHODS AND COMPOUNDS MODIFYING MITOCHONDRIAL FUNCTION

(71) Applicant: The Board of Trustees of the Leland Stanford Junior University, Stanford, CA (US)

(72) Inventors: Xinnan Wang, Redwood City, CA (US); Chung-Han Hsieh, Palo Alto, CA (US); Roeland Vanhauwaert, Oostkamp (BE)

(21) Appl. No.: 19/288,291

(22) Filed: Aug. 1, 2025

Related U.S. Application Data
(62) Division of application No. 17/640,764, now abandoned.
(60) Provisional application No. 62/896,450, filed on Sep. 5, 2019.

(51) Int. Cl. A61K 31/506; A61K 31/13; A61K 31/198; A61K 31/365; A61P 25/16; G01N 33/50; G01N 33/68

(52) U.S. Cl. CPC A61K 31/506; A61K 31/13; A61K 31/198; A61K 31/365; A61P 25/16; G01N 33/5023; G01N 33/6896; G01N 2333/914; G01N 2800/2835

(57) ABSTRACT

The invention provides methods for identifying modulators of mitochondrial function for therapeutic use in neurodegenerative disorder. The invention provides method for identifying subjects who may benefit from the therapeutic agents.

### CROSS REFERENCE TO RELATED APPLICATIONS

This application is a Divisional and claims priority to 371 application Ser. No. 17/640,764, filed Mar. 4, 2022, which claims priority to PCT Application No. PCT/US2020/049431, filed Sep. 4, 2020, which claims priority to U.S. Provisional Application No. 62/896,450, filed Sep. 5, 2019, which applications are incorporated herein in their entirety.

### REFERENCE TO ELECTRONIC SEQUENCE LISTING

The contents of the electronic sequence listing (STAN-1770DIV seqlist.xml; Size: bytes 4,876,047 bytes; and Date of Creation: Jul. 31, 2025) is herein incorporated by reference in its entirety.

### BACKGROUND

Neurons are metabolically active cells with high energy demands at locations distant from the cell body. As a result, these cells are particularly dependent on mitochondrial function, as reflected by the observation that diseases of mitochondrial dysfunction often have a neurodegenerative component. Recent discoveries have highlighted that neurons are reliant particularly on the dynamic properties of mitochondria. Mitochondria are dynamic organelles by several criteria. They engage in repeated cycles of fusion and fission, which serve to intermix the lipids and contents of a population of mitochondria. In addition, mitochondria are actively recruited to subcellular sites, such as the axonal and dendritic processes of neurons. Finally, the quality of a mitochondrial population is maintained through mitophagy, a form of autophagy in which defective mitochondria are selectively degraded. Defects in the key features of mitochondrial dynamics, such as mitochondrial fusion, fission, transport and mitophagy are associated with neurodegenerative disorder. Several major neurodegenerative disorders—including Parkinson's, Alzheimer's and Huntington's disease—involve disruption of mitochondrial dynamics.

Mitochondrial movements are tightly controlled to maintain energy homeostasis and prevent oxidative stress. Mitochondrial motility ceases prior to the initiation of mitophagy, a crucial cellular mechanism by which depolarized mitochondria are degraded through autophagosomes and lysosomes. The arrest of motility may sequester damaged mitochondria, preventing them from moving and from reintroducing damage to other healthy mitochondria.

Miro is an outer mitochondrial membrane (OMM) protein that anchors the microtubule motors kinesin and dynein to mitochondria (Glater E E, Megeath L J, Stowers R S, Schwarz T L. Axonal transport of mitochondria requires milton to recruit kinesin heavy chain and is light chain independent. The Journal of cell biology. 2006; 173:545-557; Koutsopoulos O S, Laine D, Osellame L, Chudakov D M, Parton R G, Frazier A E, Ryan M T. Human Miltons associate with mitochondria and induce microtubule-dependent remodeling of mitochondrial networks. Biochimica et biophysica acta. 2010; 1803:564-574.). This depolarization-triggered mitochondrial arrest is achieved by removal of Miro from the damaged mitochondrial surface (Wang X, Winter D, Ashrafi G, Schlehe J, Wong Y L, Selkoe D, Rice S, Steen J, LaVoie M J, Schwarz T L. PINK1 and Parkin target Miro for phosphorylation and degradation to arrest mitochondrial motility. Cell. 2011; 147:893-906.). Miro is subsequently degraded by proteasomes (Wang X, Winter D, Ashrafi G, Schlehe J, Wong Y L, Selkoe D, Rice S, Steen J, LaVoie M J, Schwarz T L. PINK1 and Parkin target Miro for phosphorylation and degradation to arrest mitochondrial motility. Cell. 2011; 147:893-906.). Evidence has shown that two PD-linked proteins, PINK1 (PTEN-induced putative kinase 1) and Parkin, act in concert to target Miro for degradation (Ashrafi G, Schlehe J S, LaVoie M J, Schwarz T L. Mitophagy of damaged mitochondria occurs locally in distal neuronal axons and requires PINK1 and Parkin. The Journal of cell biology. 2014; 206:655-670.; Liu S, Sawada T, Lee S, Yu W, Silverio G, Alapatt P, Millan I, Shen A, Saxton W, Kanao T, et al. Parkinson's disease-associated kinase PINK1 regulates Miro protein level and axonal transport of mitochondria. PLoS genetics. 2012; 8:e1002537; Wang X, Winter D, Ashrafi G, Schlehe J, Wong Y L, Selkoe D, Rice S, Steen J, LaVoie M J, Schwarz T L. PINK1 and Parkin target Miro for phosphorylation and degradation to arrest mitochondrial motility. Cell. 2011; 147:893-906.). Mutations in PINK1 or Parkin are tied to rare forms of recessive early-onset PD.

Altered mitochondrial transport is one of the pathogenic changes in major adult-onset neurodegenerative diseases. In mutant LRRK2GS2019 cells, the mitochondrial outer membrane protein Miro is stabilized and remains on damaged mitochondria for longer than normal, prolonging active transport and inhibiting mitochondrial degradation (Hsieh et, al, 2016). Miro degradation and mitochondrial motility are also impaired in sporadic PD subjects. Prolonged retention of Miro, and the downstream consequences that ensue, may constitute a central component of PD pathogenesis.

Therefore, there is a need for novel therapeutic approaches and methods to identify modulators of Miro retention and degradation that promote arrest of mitochondrial motility of damaged mitochondria and restoration of mitochondrial dynamics in neurodegenerative disease.

### SUMMARY

Methods and compositions are provided for the monitoring, treatment, and screening for a neurodegenerative disorder, e.g., Parkinson's Disease, in a subject. Aspects of the methods include administering MIRO1 reducer to a subject having Parkinson's Disease. Also provided are companion diagnostic assays to determine if a subject is suitable for treatment with a MIRO1 reducer, and treating the subject in accordance with the results.

In one aspect, provided is a method to determine the MIRO1 status of a subject comprising measuring the Miro1 response to mitochondrial depolarization using biochemical assays, western blotting or ELISA, to determine if a subject is deficient in the removal of MIRO1 following depolarization, wherein a subject deficient in the removal of MIRO1 following depolarization is selected for treatment by administration of a MIRO1 reducer. Determining the MIRO1 status can comprise detecting MIRO1 level in a subject, and comparing the MIRO1 level to a control MIRO1 level in a control subject. In some embodiments, detecting MIRO1 level comprises detecting MIRO1 in a tissue sample, such as a skin fibroblast, in the subject. In some embodiments, the method comprises an assay described in any one of the Examples.

Provided herein is a method to screen candidate agents for activity to reduce MIRO1 level, the method comprising: (a) contacting a candidate agent and a sample from a subject deficient in or suspected of being deficient in the removal of MIRO1, (b) measuring the MIRO1 level in the sample, (c) comparing the MIRO1 level in a sample with a control MIRO1 level, and (d) assessing the activity of the candidate agent if the MIRO1 level in the sample is lower than the control MIRO1 level.

Provided here in a method of treating a neurodegenerative disorder comprising administering to a subject in need thereof a Miro-reducing agent, wherein the Miro-reducing agent reduces Miro in sporadic Parkinson's disease cells with depolarized mitochondria by three standard deviations or more as compared to reduction of Miro in control depolarized sporadic Parkinson's disease cells with depolarized mitochondria not contacted with the Miro-reducing agent. The healthy cells can be healthy cells with depolarized mitochondria, wherein the healthy cells are contacted with a mitochondrial depolarizing agent.

Provided herein is a method of treating a neurodegenerative disorder comprising administering to a subject in need thereof a Miro-reducing agent, wherein the Miro-reducing agent reduces Miro in Parkinson's disease cells with depolarized mitochondria by three standard deviations or more as compared to reduction of Miro in control depolarized sporadic Parkinson's disease cells with depolarized mitochondria not contacted with the Miro-reducing agent.

Provided herein is a method of treating a neurodegenerative disorder comprising administering to a subject in need thereof a Miro-reducing agent, wherein: (a) the Miro-reducing agent reduces Miro in Parkinson's disease cells with depolarized mitochondria by two standard deviations or more as compared to reduction of Miro in control Parkinson's disease cells with depolarized mitochondria not contacted with the Miro-reducing agent; and (b) the Miro 1 reducing agent reduces Miro in Parkinson's disease cells with non-depolarized mitochondria by one standard deviation or less as compared to reduction of Miro in control Parkinson's disease cells with non-depolarized mitochondria not contacted with the Miro-reducing agent.

The Parkinson's disease cells can be Parkinson's disease cells with depolarized mitochondria contacted with a mitochondrial depolarizing agent. The Parkinson's disease cells can be sporadic Parkinson's disease cells. The Parkinson's disease cells can be fibroblasts.

Provided herein is a method of treating a neurodegenerative disorder comprising administering to a subject in need thereof a Miro-reducing agent, wherein the Miro-reducing agent reduces Miro in the following assay: (a) fibroblast cells from a sporadic Parkinson's disease patient are plated into wells of an array (b) 24 hours after step (a), a candidate agent is added to a test well and the candidate agent is not added to a control well; (c) 10 hours after step (b), FCCP is added to the test well and control well; (d) 14 hours after step (c) cells of the test well and control well are fixed with ice-cold 90% methanol; and (e) the cells of the test well and control well are immunostained with anti-Miro and 4′,6-diamidino-2-phenylindole, dihydrochloride (DAPI) and imaged with a confocal microscope to measure Miro intensity/cell for those images of the test well and control well; wherein when the candidate agent reduces Miro in the test well by three standard deviations or more relative to the control well, the candidate agent is a Miro-reducing agent.

Provided herein is a method of treating a neurodegenerative disorder comprising administering to a subject in need thereof a Miro-reducing agent, wherein the Miro-reducing agent reduces Miro in sporadic Parkinson's disease cells according to the following assay: (a) fibroblast cells from a sporadic Parkinson's disease patient are plated into wells of an array; (b) 24 hours after step (a), a candidate agent is added to a first test well and the candidate agent is not added to a first control well; (c) 10 hours after step (b), FCCP is added to a first test well and first control well; (d) 14 hours after step (c), cells of the first test well and first control well are fixed with ice-cold 90% methanol; and (e) the cells of the first test well and first control well are immunostained with anti-Miro and 4′,6-diamidino-2-phenylindole, dihydrochloride (DAPI) and imaged with a confocal microscope to measure Miro intensity/cell for those images of the first test well and first control well; and wherein the Miro-reducing agent does not reduce Miro in sporadic Parkinson's disease cells according to the following assay: (a1) fibroblast cells from a sporadic Parkinson's disease patient are plated into wells of an array; (b1) 24 hours after step (a1), the candidate agent is added to a second test well and is not added to a second control well; (c1) 14 hours after step (b1), cells of the second test well and second control well are fixed with ice-cold 90% methanol; and (d1) the cells of the second test well and second control well are immunostained with anti-Miro and 4′,6-diamidino-2-phenylindole, dihydrochloride (DAPI) and imaged with a confocal microscope to measure Miro intensity/cell for those images of the second test well and second control well; wherein when the candidate agent reduces Miro in the first test well by two standard deviations or more relative to the first control well, and the candidate agent reduces Miro in the second test well by less than one standard deviation relative to the second control well, the candidate agent is a Miro-reducing agent.

In certain embodiments, the neurodegenerative disorder is Parkinson's Disease (PD). In some such embodiments, the PD is a familial form of PD, e.g. a PTEN-induced putative kinase 1 (PINK-1)-associated form of PD, a Parkin-associated form of PD, an LRRK2-associated form of PD, an alpha-Synuclein (SNCA)-associated form of PD, an E3 ligase (Parkin)-associated PD, a GBA-associated PD, a ubiquitin carboxy-terminal hydrolase L1 (UCHL1)-associated form of PD, a parkinson protein 7 (PARK7, DJ-1) associated form of PD, an ATP13A2-associated form of PD, a phospholipase A2, group VI (PLA2G6)-associated form of PD, a DnaJ (Hsp40) homolog, subfamily C, member 6 (DNAJC6, PARK19)-associated form of PD; a eukaryotic translation initiation factor 4 gamma, 1 (EIF4G1, PARK18)-associated form of PD; a F-box protein 7 (FBXO7)-associated form of PD; a GRB10 interacting GYF protein 2 (GIGYF2)-associated form of PD; a HtrA serine peptidase 2 (HTRA2)-associated form of PD; a synaptojanin 1 (SYNJ1)-associated form of PD; and a vacuolar protein sorting 35 homolog (VPS35)-associated form of PD. In other such embodiments, the PD is a sporadic form of Parkinson's Disease, for example, it is associated with a sporadic mutation in one of the aforementioned genes. In certain such embodiments, the MIRO1 reducer is administered to the midbrain and/or putamen of the subject.

In some embodiments, an assay is provided for determining the MIRO1 status of a subject. In some embodiments an assay is performed on a population of patient cells, conveniently fibroblasts are used, to identify the MIRO1 phenotype of a subject. Miro1 is localized on the mitochondrial surface and mediates mitochondrial motility. Miro1 is removed from depolarized mitochondria to facilitate their clearance via mitophagy. Measuring the Miro1 response to mitochondrial depolarization using biochemical assays, ELISA, etc. shows that a high percentage of Parkinson's Disease subjects are deficient in the removal of MIRO1 following depolarization.

As described in the Examples herein, MIRO1 status correlates with the presence of certain neurodegenerative disorders. For instance, an elevated level of MIRO1 is associated with Parkinson's disease.

The Miro-reducing agent can reduce intracellular calcium levels by 20% or more. The Miro-reducing agent can have a molecular weight of from 100 to 2000 Daltons. The Miro-reducing agent can be an antibody, peptide, or protein or portion of one thereof. The Miro-reducing agent can bind to an EF hand protein. The Miro-reducing agent can be a calcium channel blocker. The calcium channel blocker can be an L-type and N-type calcium channel blocker. The subject can have elevated Miro. The subject can be otherwise asymptomatic for the neurodegenerative disorder. The neurodegenerative disorder can be Parkinson's disease.

Provided herein is a method for selecting a subject for treatment with a therapeutic agent for a neurodegenerative disorder, comprising: (a) collecting cells from the subject and evaluating a first control portion of the cells for the pre-depolarization Miro level in the cells; (b) contacting a second test portion of the cells with a depolarizing agent; and (c) evaluating the post-depolarization Miro level in the second test portion of cells contacted with the depolarizing agent and comparing the Miro level to the pre-depolarization Miro level in the first control portion of cells; wherein when the post-depolarization Miro level in the second test portion of cells is reduced by 40% or less relative to the pre-depolarization Miro level in the first control portion of cells, the subject is treated with a therapeutic agent for neurodegenerative disorder. Provided herein is a method for selecting a subject for treatment with a therapeutic agent for a neurodegenerative disorder, wherein when the post-depolarization Miro level in the second test portion of cells is reduced by 10% to 50% relative to the pre-depolarization Miro level in the first control portion of cells, the subject is treated with a therapeutic agent for neurodegenerative disorder.

The neurodegenerative disorder can be Parkinson's disease. The subject can be asymptomatic for Parkinson's disease. The therapeutic agent can be selected from levodopa and dopamine antagonists. The therapeutic agent can be a Miro-reducing agent. The therapeutic agent can have a molecular weight of from 100 to 2000 Daltons. The therapeutic agent can an antibody, peptide, or protein or portion of one thereof. The therapeutic agent can bind to an EF hand protein. The therapeutic agent can be a calcium channel blocker. The calcium channel blocker can be an L-type and N-type calcium channel blocker.

Provided herein is a method of improving a condition of aging, comprising administering to a subject in need thereof a Miro-reducing agent. The condition of aging can be selected from memory impairment, muscle degeneration, arthritis, cardiovascular disease, osteoporosis, glaucoma, dementia, macular degeneration, and cataract. The Miro-reducing agent can be administered prophylactically.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Before the present methods and compositions are described, it is to be understood that aspects of the invention are not limited to particular method or composition described, as such may, of course, vary. It is also to be understood that the terminology used herein is for the purpose of describing particular embodiments only, and is not intended to be limiting, since the scope of the present invention will be limited only by the appended claims.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1F. Miro1 Response to CCCP in Fibroblasts. FIG. 1A: Schematic representation of our readouts. FIGS. 1B-1C: Examples of the readouts using healthy-1, PD-2, and risk-2. FIG. 1D: Heat maps show the relative mitochondrial protein levels. The intensity of each band in the mitochondrial fraction is normalized to that of the mitochondrial loading control VDAC from the same blot and expressed as a fraction of the mean of Healthy-1 with DMSO treatment; this control was included in every experiment. Mean values are imported into heat maps. n=3-35. FIGS. 1E-1F: ELISA of MiroL. n=4 with duplicates each time. Comparison within the same subject. Throughout *: P<0.05; **: P<0.01; ***: P<0.001.

FIGS. 2A-2H show Miro1 ratio (CCCP/DMSO) vs. different variables. FIG. 2A: healthy subjects and Parkinson's disease subjects (“PD”); FIG. 2B: male and female subjects; FIG. 2C: age at sampling (years); FIG. 2D: PD onset age (years); FIG. 2E: PD progression (years with PD); FIG. 2F: UPDRS; FIG. 2G: Hoehn and Yahr scale of PD subjects; FIG. 2H: mini-mental state examination in PD subjects.

FIGS. 3A-3G: FIG. 3A shows relative β-actin levels or Miro1 levels (ng/ml) in subjects; FIG. 3B shows absorbance at 450 nm vs. β-actin levels; FIG. 3C shows relative β-actin levels vs. capture antibody; FIG. 3D shows absorbance at 450 nm vs. Miro1 levels; FIG. 3E shows relative Miro1 levels vs. capture antibody; FIG. 3F shows absorbance at 450 nm vs. Miro1 (ng/ml); FIG. 3F shows Miro1 (ng/ml) vs. capture antibody.

### DETAILED DESCRIPTION

In one aspect, the disclosure herein provides a method of diagnosing a neurodegenerative disorder, e.g., Parkinson's disease, comprising detecting Miro, e.g., Miro1, in a subject. In certain embodiments, the Miro1 level in a subject, e.g., Miro1 level in a tissue sample from a subject, is detected using an assay described herein. The Miro1 level in a subject can be compared to a control Miro1 level in a control subject in order to determine the presence or absence of a neurodegenerative disorder. For example, a Miro1 level that is higher, such as 20% or more, or 30% or more, than a control Miro1 level, it can indicate the presence of Parkinson's disease. If the presence of a neurodegenerative disorder is indicated, the subject can be treated for the neurodegenerative disorder, such as with a treatment known in the art or described herein.

In one aspect, the disclosure herein provides a method of treating a neurodegenerative disorder, e.g., Parkinson's disease, comprising administering to a subject in need thereof a Miro-reducing agent, e.g., a Miro1-reducing agent. In certain embodiments, the Miro-reducing agent is identified using an assay described herein. In certain embodiments, the Miro-reducing agent reduces Miro, e.g., Miro1, levels in cells, for example, cells from a subject deficient in or suspected of being deficient in the removal of Miro1. In certain embodiments, the Miro-reducing agent reduces Miro, e.g., Miro1, levels in cells to a level consistent with Miro, e.g., Miro1, levels in control cells.

In certain embodiments, the disclosure provides methods of treating a subject with a neurodegenerative disorder, e.g., Parkinson's disease, wherein the subject is diagnosed with Parkinson's disease using a method described herein. Methods of treating neurodegenerative disorder with Miro-reducing agents are described below.

In certain embodiments, the disclosure provides a method of identifying Miro1 reducing agents. In certain embodiments, the disclosure provides a method of screening candidate agents for use in the treatment of a neurodegenerative disorder, e.g., Parkinson's disease, using an assay described herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the subject, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalia subject for whom diagnosis, treatment, or therapy is desired, particularly humans.

It must be noted that as used herein and in the appended claims, the singular forms “a,” “and,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “candidate agent,” “test agent,” “agent,” “substance,” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds.

Mitochondrial Rho (Miro) is a small GTPase known for its functions in mitochondria transport and homeostasis. Miro is a highly conserved protein across unicellular and multicellular eukaryotes. Miro1 and Miro2 are conserved single-pass transmembrane integral proteins with N-terminal regions exposed to the cytosolic side and consist of two GTPase domains separated by a pair of canonical EF hands. Miro also contains a C-terminal hydrophobic domain that allows membrane insertion. Miro GTPases are localized to the mitochondrial outer membrane and play critical roles in intracellular mitochondria movement in metazoans, particularly over long distances along microtubule tracts in neurons. Miro is also known to play a role in mediating intercellular transport of mitochondria between cells via tunneling nanotubes. Miro mediates bidirectional mitochondrial movement along microtubule tracts by engaging both kinesin and dynein. Miro engages with microtubule tracks via the cargo adaptors of the Milton/Trak family. Miro also transports mitochondria along the actin tracks using the mitochondrial actin motor Myo19. Miro is also involved in peroxisomal distribution through binding with Peroxin 26. In addition, Miro also has roles in mitochondrial fusion and fission dynamics through its modulation of the mitochondrial dynamin Drp1 and interactions with the mitochondrial fusion proteins mitofusins 1 and 2.

Miro turnover is regulated by the PTEN-induced putative kinase 1 (PINK1)/Parkin pathway. PINK1 phosphorylates Miro, thus promoting its interaction with the E3 ubiquitin ligase Parkin. Parkin promotes Miro ubiquitination and degradation, which effectively arrests axonal transport of damaged mitochondria. On the other hand, PINK1 phosphorylated Miro also recruits Parkin to the damaged mitochondria, which then tags these for mitophagic destruction.

Miro is located in the outer mitochondrial membrane and anchors the microtubule motors kinesin and dynein to mitochondria. Upon mitochondrial damage caused by mitochondrial depolarization, mitophagy is initiated in the cell. Mitochondrial motility ceases prior to the initiation of mitophagy, a cellular mechanism, by which depolarized mitochondria are degraded through autophagosomes and lysosomes. The arrest of motility may sequester damaged mitochondira, preventing them from moving on and from reintroducing damage to other healthy mitochondria. This depolarization-triggered mitochondrial arrest is achieved by removal of Miro from the damaged mitochondrial surface and subsequent degradation by proteasomes. However, a notable impairment in the process of Miro degradation and subsequent clearance of damaged mitochondria is observed in skin fibroblasts from both familial and sporadic PD subjects.

The EF hands of Miro mediate Ca²⁺-dependent arrest of bidirectional mitochondrial transport. Mitochondrial transport is mediated by linking Miro to KIF5. Ca²⁺ binding detaches KIF5 from mitochondria or by turning “off” KIF5 engagement with microtubules. Ca²⁺ binding to the EF hands triggers the direct interaction of the motor domain with Miro, thus preventing the motor from engaging with MTs.

As used herein “Miro’ can refer to any one or all of the following: family members, isoforms, homologs, paralogs, mutants, alleles, variants, derivatives, fragments, species, coding and noncoding sequences, sense and antisense polynucleotide strands, etc. Miro refers to human sequence Miro, such as the complete amino acid sequence of human Miro isoforms having Uniprot Identifiers Q8IXI2-1, Q8IXI2-2, Q8IXI2-3, Q8IXI2-4, Q8IXI2-5, Q8IXI2-6 or Q8IXI2-7. The human Miro sequence may differ from human Miro isoforms of Uniprot identifiers Q8IXI2-1, Q8IXI2-2, Q8IXI2-3, Q8IXI2-4, Q8IXI2-5, Q8IXI2-6 or Q8IXI2-7. by having, for example, conserved mutations or mutations in non-conserved regions and the miro has substantially the same biological function as the human Miro isoforms of Q8IXI2-1, Q8IXI2-2, Q8IXI2-3, Q8IXI2-4, Q8IXI2-5, Q8IXI2-6 or Q8IXI2-7 such as binding to mitochondria. Miro is also referred to as used RHOT1, and Mitochondrial Rho GTPase1. Miro includes isoforms of Miro such as Miro1 and Miro2.

A particular Miro sequence can be at least 90%, at least 95%, or even at least 96%, at least 97%, at least 98%, or at least 99% identical in amino acid sequence to Miro of Uniprot identifiers Q8IXI2-1, Q8IXI2-2, Q8IXI2-3, Q8IXI2-4, Q8IXI2-5, Q8IXI2-6 or Q8IXI2-7. In certain embodiments, a human Miro sequence can differ by more than 10 amino acid differences from the Miro sequence of Uniprot identifiers Q8IXI2-1, Q8IXI2-2, Q8IXI2-3, Q8IXI2-4, Q8IXI2-5, Q8IXI2-6 or Q8IXI2-7. In certain embodiments, the human Miro may display no more than 5, or even no more than 4, no more than 3, no more than 2, or no more than 1 amino acid difference from the Miro sequence of Uniprot identifiers Q8IXI2-1, Q8IXI2-2, Q8IXI2-3, Q8IXI2-4, Q8IXI2-5, Q8IXI2-6 or Q8IXI2-7. Percent identity can be determined as described herein.

An “isoform” of a protein can be, e.g., a protein resulting from alternative splicing of a gene expressing the protein, or a degradation product of the protein. “Sequence homology”, refers to a nucleotide-to-nucleotide or amino acid-to-amino acid correspondence of two polynucleotides or polypeptide sequences, respectively. As used herein, “sequence identity” or “identity” refers, in the context of two nucleic acid sequences or amino acid sequences, to the residues in the two sequences that are the same when aligned for maximum correspondence over a specified comparison window.

As used herein, “percent sequence identity” means the value determined by comparing two optimally aligned sequences over a comparison window, wherein the portion of the polynucleotide or polypeptide sequence in the comparison window may comprise additions or deletions, i.e., gaps, compared to the reference sequence which does not comprise additions or deletions can be used for optimal alignment of the two sequences. The percentage can be calculated by: determining the number of positions at which the identical nucleotide or amino acid occurs in both sequences to yield the number of matched positions; dividing the number of matched positions by the total number of positions to give in the comparison window; and multiplying the result by 100 to determine the percentage of sequence identity.

Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters.

The “percent identity” between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program can bebased on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program can define identity as the number of identical aligned symbols (i.e., nucleotides or amino acids), divided by the total number of symbols in the shorter of the two sequences. The program may be used to determine percent identity over the entire length of the sequences being compared. Default parameters can be provided to optimize searches with short query sequences, for example, with the blastp program. The program can also allow use of an SEG filter to mask-off segments of the query sequences as determined by the SEG program of Wootton and Federhen, Computers and Chemistry 17: 149-163 (1993). High sequence identity can include sequence identity in ranges of sequence identity of approximately 80% to 99% and integer values there between.

A “homolog” can refer to any sequence that has at least 50%, at least 55%, at least 60%, at least 65%, at least 70%, at least 75%, at least 80%, at least 85%, at least 90%, at least 95%, at least at least 96%, at least 97%, at least 98%, at least 99%, or at least 99.5% sequence homology to another sequence. In certain embodiments, a homolog has from 70% to 99%, from 80% to 99%, from 85% to 99%, from 90% to 99% or even from 95% to 99% sequence homology to another sequence. In some cases, the homolog can have a functional or structural equivalence with the native or naturally occurring sequence. In some cases, the homolog can have a functional or structural equivalence with a domain, a motif or a part of the protein, that is encoded by the native sequence or naturally occurring sequence.

Homology comparisons may be conducted with sequence comparison programs. Computer programs may calculate percent (%) homology between two or more sequences and may also calculate the sequence identity shared by two or more amino acid or nucleic acid sequences. Sequence homologies may be generated by any of a number of computer programs, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60).

Percent homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments can be performed over a relatively short number of residues.

In an otherwise identical pair of sequences, one insertion or deletion may cause the following amino acid or nucleotide residues to be put out of alignment, thus potentially resulting in a large reduction in % homology when a global alignment is performed. Consequently, the sequence comparison method can be designed to produce optimal alignments that take into consideration possible insertions and deletions without unduly penalizing the overall homology or identity score. This can be achieved by inserting “gaps” in the sequence alignment to try to maximize local homology or identity.

Calculation of maximum % homology can make use of optimal alignment, taking into consideration gap penalties. BLAST 2 Sequences is another tool that can be used for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health).

Homologous sequences can also have deletions, insertions or substitutions of amino acid residues which result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. Sets of conserved amino acids may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218).

“Miro-reducing agent”, e.g., a MIRO1 reducer, refers to any agent that decreases the level of a Miro protein, or a homolog thereof, in cells with depolarized mitochondria. In an exemplary embodiment, a Miro-reducing agent may decrease at least one biological activity of a Miro protein in a cell with depolarized mitochondria. Exemplary biological activities of Miro include promoting mitochondrial transport, mitophagy, microtubule binding, mitochondrial fission and fusion among others. A Miro-reducing agent can be, for example, a small molecule, a peptide, an aptamer, a protein or a functional fragment of a protein. A functional fragment of a protein, as used here, refers to all or part of the molecular elements of a protein which affect a specified function such as protein binding, signal transduction etc.

In some embodiments, a MIRO1 reducer will inhibit the level or biological activity of MIRO1 by 20% or more, for example, 30% or more, 40% or more, or 50% or more, sometimes 60% or more, 70% or more, or 80% or more, e.g. 90%, 95%, or 100%, relative to an untreated control not contacted with the reducer. A reducer may be validated as such by any convenient method in the art for detecting the level and/or activity of MIRO1 in the presence versus absence of the MIRO1 reducer.

For example, the level and/or the phosphorylation state of a Miro protein (Ser156, Thr298 or Thr299 of Miro1 and Miro2, see, e.g., Wang et al. Cell 2011, 147(4): 893-906) may be detected, for example by immunoprecipitation with a mitochondrial transport protein-specific antibody followed by Western blotting with a phospho-specific or a general antibody, where an increase in phosphorylation of Miro proteins and/or a decrease of total Miro protein levels, or a decrease in phosphorylation of Khc following contact with the agent may indicate that the agent will treat Parkinson's Disease. As another example, the level and/or the ubiquitination of a Miro protein may be detected, for example by immunoprecipitation with a mitochondrial transport protein-specific antibody followed by Western blotting with a ubiquitin-specific antibody, where an increase in ubiquitination following contact with the candidate agent indicates that the agent will treat Parkinson's Disease. As another example, the ability of the target mitochondrial protein to transport mitochondria within a cell may be assessed by, for example, treating cultured cells (e.g., neurons) with the MIRO1 reducer and observing the transport of mitochondria in the cells as compared to cells not treating with the MIRO1 reducer, e.g., using live cell imaging techniques (see, e.g., Brickley and Stephenson J. Biol Chem 286(20): 18079-92 (2011); Misko et al. J Neurosci 30(19): 4232-40 (2010); Russo G J et al. J. Neurosci 29(17):5443-55 (2009)). As another example, because the formation of a complex between Miro (e.g., Miro 1 and 2), TRAK (e.g., TRAK1 and 2), and Khc is essential for mitochondrial transport in neurons (see e.g., Brickley and Stephenson J. Biol Chem 286(20): 18079-92 (2011)), the effect of MIRO1 reducer on Miro function may be assessed by assessing the ability of Miro, TRAK and Khc to form a complex in the presence of the MIRO1 reducer. Such an assessment can be performed using any technique to determine protein-protein interaction including, but not limited to, co-immunoprecipitation and affinity purification techniques. In specific embodiments, the ability is assessed in a cell having a familial PD mutation, e.g. a PINK1 or LRRK2 mutation.

“Mitochondrial depolarization” is the process in which the membrane potential of the mitochondria changes in the depolarizing direction from the resting potential, from negative to positive. Normal, mild calcium influx from cytosol into the mitochondrial matrix causes transient depolarization that is corrected by pumping out protons. Chemically induced mitochondrial depolarization provides a suitable assay for determining the effect of damage to mitochondria.

A number of agents are known and used experimentally to cause mitochondrial depolarization and can be used in the methods of the invention for that purpose. Such agents are generally in the class of mitochondrial uncouplers or uncoupling agents that disrupt oxidative phosphorylation in mitochondria by dissociating the reactions of ATP synthesis from the electron transport chain. The result is that the mitochondrion expends energy to generate a proton motive force, but the proton motive force is dissipated before the ATP synthase can recapture this energy and use it to make ATP.

The concentration of agent and time of exposure is sufficient to uncouple or depolarize the mitochondria. For example, CCCP at a concentration of from about 10-100 μM, for example from about 20 to 80 μM, from about 30 to 50 μM is sufficient. Dosage of other agents may provide for activity comparable to these concentrations of CCCP. The cells are incubated for a period of time sufficient to depolarize mitochondria and initiate clearance, e.g. at least about 1 hour, at least about 2 hours, and usually not more than about 24 hours, and may be from about 1 to 24 hours, from about 2 to 20 hours, from about 3 to 18 hours, from about 4 to 14 hours, from about 5 to 10 hours, and may be from about 4 to 8 hours.

Carbonyl cyanide p-trifluoro-methoxyphenyl hydrazone (FCCP) and carbonylcyanide-3-chlorophenylhydrazone (CCCP) are mitochondrial uncouplers frequently used in research. These molecules are lipophilic weak acids that act as protonophores. Due to their hydrophobic nature, these compounds can easily traffic across biological membranes and allow the protons to cross these membranes. Other molecules suitable for this purpose include 1,3-bis(3,5-dichlorophenyl)urea; dodecyltriphenylphosphonium; dinitrophenol; BAM15 ((2-fluorophenyl)6-[(2-fluorophenyl)amino](1,2,5-oxadiazolo[3,4-e]pyrazin-5-yl)amine), which is a mitochondria-specific protonophore uncoupler that possesses a similar potency to FCCP or DNP; FR58P1 (a bromoalkyl ester of a hydroquinone derivative) is another mitochondrial protonophore.

Other mitochondria-specific uncouplers include MitoFluo, which is a conjugate of a triphenylphosphonium cation to fluorescein, acting as a fluorescent uncoupler that accumulates preferentially in mitochondria; dodecyltriphenylphosphonium (C12TPP), which operates as a fatty acid anion carrier and facilitates fatty acid cycling across the membrane and thus mitochondria uncoupling; the Rhodamine 19 butyl ester C4R1, which acts as a mild mitochondrial uncoupler; and MitoPhotoDNP, which is a fusion of DNP, the o-nitrobenzyl group (a photoactivable group), and triphenylphosphonium. Bupivacaine, a local anesthetic agent, can act partially as a protonophore but also presents an inhibitory effect on state 3-respiration by altering the mitochondrial proton pump stoichiometry. Weak C—H acids, such as ortho-carborane (1,2-C₂B₁₀H₁₂), also have global uncoupling properties when used at concentrations comparable to FCCP (10 μM range).

Affinity Assays, which are often immunoassays, are an assay or analytic procedure that relies on the binding of the target molecule, i.e. Miro1, to receptors, antibodies or other macromolecules. A detection method is used to determine the presence and extent of the binding complexes that are formed. Many formats for such assays are known and used in the art, and are suitable for detection of Miro1 degradation following mitochondrial uncoupling or depolarization. In some embodiments, the assay format is suitable for high-throughput analysis.

Included in suitable assay formats are immunoassays that utilize antibodies specific for Miro1. Suitable antibodies for this purpose are known and commercially available as polycloncal or monoclonal compositions, e.g. from Invitrogen, including monoclonals CL1095, CL1083; from Sigma Aldrich including clone 4H4, Santa Cruz Biotechnology Anti-Rho T1 Antibody (A-8); and the like.

Assays of interest include, for example, Western blots; immunohistochemistry; immunoprecipitation; etc., and particularly include immunoassays such as enzyme-linked immunosorbent assay (ELISA), radioimmunoassay (RIA); enzyme immunoassay (EIA).

Enzyme-linked immunosorbent assays (ELISAs) are used to qualitatively and quantitatively analyze the presence or concentration of a particular soluble antigen such as Miro1, in liquid samples, such as cell lysates. These assays generally make use of the ability of multiwell plates or others to bind antibodies which trap the cognate antigen. Usually a colorimetric endpoint that can be detected via absorbance wavelength and quantitated from a known standard curve of antigen or antibody dilutions is used. The detection antibody is often labelled with an enzyme such as horseradish peroxidase or alkaline phosphatase, or a fluorescent tag, or an electrochemiluminescent label or through an intermediary label such as biotin.

Common ELISA formats include the sandwich ELISA, so named because the analyte is “sandwiched” between two different antibodies. The capture substrate in this format is a capture antibody, often a monoclonal antibody, to increase the specificity of the assay and reduce background noise. The analyte is bound to the capture antibody, then detected by binding to a detection antibody. A variation of sandwich ELISA assay, called Single-Molecule Assay (Simoa), uses beads are coated with a capture antibody; each bead is bound to either one or zero target molecule, and individual beads are detected with another antibody (detection antibody) and a labeling enzyme.

Other ELISA formats include indirect ELISA, where the capture substrate is the specific antigen that is being tested and the detection step is mediated by a primary antibody and an enzyme-conjugated secondary antibody which is reactive against the primary antibody. Thus, the primary antibody that recognizes the antigen is not labeled. In a direct ELISA the capture substrate is the specific antigen that is being tested, and the enzyme that catalyzes the color-change reaction is conjugated to the antigen detector antibody.

Immuno-PCR (I-PCR) is a technique that combines the sensitivity of the nucleic acid amplification by PCR with the specificity of the antibody-based assays resulting in an increase of the detection sensitivity.

### Methods of Screening

In some aspects of the invention, methods are provided for screening candidate agents for activity in treating Parkinson's Disease in a subject having Parkinson's Disease. To that end, it has been shown that mitochondrial transport proteins, for example, Miro proteins, trafficking kinases, and the kinesin heavy chain, promote the development or progression of Parkinson's Disease or the symptoms thereof. Accordingly, screening for candidate agents that inhibit the expression or activity of mitochondrial transport proteins in cells should identify agents that will be useful in treating Parkinson's Disease in subjects. In particular agents are tested for the ability to reduce levels of Miro1 following mitochondrial depolarization, and preferably without a substantial reduction in basal Miro1 levels.

Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection.

In one embodiment, to identify agents that alter Miro1 degradation; a cell, e.g. a viable cell or population of cells, is contacted with an agent to be tested; alternatively, the protein can be contacted directly with the agent to be tested. The level (amount) of activity is assessed either directly or indirectly, and is compared with the level of activity in a control, e.g. in the absence of the agent to be tested. If the level of the activity in the presence of the agent differs, by an amount that is statistically significant, from the level of the activity in the absence of the agent, then the agent is an agent that alters the activity.

This invention further pertains to novel agents identified by the above-described screening assays. Accordingly, it is within the scope of this invention to further use an agent identified as described herein in the methods of treatment described herein. For example, an agent identified as described herein can be used to alter Miro1 degradation with the agent identified as described herein.

For example, in screening assays for biologically active agents, cells expressing the mitochondrial transport protein of interest are contacted with a candidate agent of interest and the effect of the candidate agent on the expression or function of the mitochondrial transport protein is assessed by monitoring one or more mitochondria-associated parameters. In particular the activity of a candidate agent can be assessed by determining the level of Miro1 following mitochondria depolarization.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may comprise contacting a cell that expresses mitochondrial transport proteins with a candidate agent; and comparing the mitochondria-associated parameter to the mitochondria-associated parameter in a cell that expresses the mitochondrial transport proteins but was not contacted with the candidate agent, wherein a difference in the parameter in the cell contacted with the candidate agent indicates that the candidate agent will treat the Parkinson's Disease.

One example of a mitochondria-associated parameter that may be quantified when screening for agents that will treat Parkinson's Disease would be the phosphorylation state of a Miro protein (Ser156, Thr298 or Thr299 of Miro1 and Miro2), a TRAK protein, or Khc (Ser residues, see, e.g., Lee and Hollenbeck J Biol Chem. 1995 270(10):5600-5), by for example, immunoprecipitation with mitochondrial transport protein-specific antibodies followed by Western blotting with phospho-specific and general antibodies where an increase in phosphorylation of Miro proteins and/or a decrease in total Miro levels, or a decrease in phosphorylation of Khc following contact with the candidate agent indicates that the candidate agent may treat Parkinson's Disease. Another example of a parameter would be the state of ubiquitination of a Miro protein, a TRAK protein, or Khc, by, for example, immunoprecipitation with mitochondrial transport protein-specific antibodies followed by Western blotting with ubiquitin-specific antibodies, where an increase in ubiquitination following contact with the candidate agent indicates that the agent will treat Parkinson's Disease. Yet another example would be the rate at which mitochondria are transported around the cell, measurable by, for example live cell imaging techniques, where a decrease in the rate of transport after contacting the cell with candidate agent indicates that the agent will treat Parkinson's Disease. Another example would be the length of the mitochondria in a cell, where a decrease in the length of the mitochondria after contacting the cell with candidate agent indicates that the agent will treat Parkinson's Disease. Other output parameters could include those that are reflective of the ability of Miro proteins, TRAK proteins, and khc to form a complex (see e.g., Brickley and Stephenson J. Biol Chem 286(20): 18079-92 (2011)) in the presence of the MIRO1 reducer, which may be assessed by, for example, co-immunoprecipitation or affinity purification techniques, where a decrease in complex formation after contacting the cell with candidate agent indicates that the agent will treat Parkinson's Disease. In some instances, one parameter is measured. In some instances, multiple parameters are measured.

All cells comprise mitochondria, and thus, any cell may be used in the subject screening methods. In some instances, the cell is a cell type that is typically affected by Parkinson's Disease, e.g. a muscle cell or a neuron, e.g. a motor neuron. In certain instances, the cell comprises a genetic mutation, i.e. a mutation in a nuclear or mitochondrial gene, which is associated with Parkinson's Disease. In some instances, the cell may be acutely cultured from a subject that has Parkinson's Disease.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

Antibody and small molecules may be screened using a variety of methods to detect a change in MIRO1 level in vitro and in vivo. e.g., ELISA assays, etc. These methods include, but are not limited to, methods that measure binding affinity to a target, biodistribution of the compound within an animal or cell, or compound mediated cytotoxicity. As a first test, the antibody or small molecule may be tested to establish interaction with a target which signals the level of MIRO1 to change. After selective binding is established, the candidate antibody may be tested for appropriate activity in an in vivo model. These and other screening methods known in the art provide information on the ability of a compound to bind to, modulate, or otherwise interact with the specified target and are a measure of the compound's efficacy. In some embodiments, the method of screening comprises an assay described in any one of the Examples.

### Methods of Diagnosis

In various aspects, a subject is screened to determine if the subject is suffering from or prone to a neurodegenerative disorder such as Parkinson's disease. The screening methods comprise behavioral, biophysical, biochemical, and imaging assays and observations as well as questionnaires to determine if the subject is at risk for or is suffering from the early stages of a neurodegenerative disorder (e.g., Parkinson's disease). Biophysical and behavioral observations, such as physical examination of a subject for outward symptoms of disease can be evaluated independently, or combined with questionnaires and biochemical/imaging assays. Each individual assay can also be utilized independently or combined with biophysical evaluations or other tests that are known in the art and associated with a particular neurodegenerative disorder/disease. Examples of biochemical assays include genetic screens for mutations and/or polymorphisms (e.g., SNPs analysis, short tandem repeat analysis), biomarker-based assays, protein expression assays, immunohistochemistry assays or any combinations thereof. Material for biochemical assays can be sampled from all bodily fluids and tissues. Commonly employed bodily fluids include but are not limited to blood, serum, plasma, saliva, urine, gastric and digestive fluid, tears, stool, semen, vaginal fluid, interstitial fluids derived from tumorous tissue, and cerebrospinal fluid. Methods of obtaining samples of bodily tissue and fluids include but are not limited to biopsy, cheek swabbing, nose swabbing, rectal swabbing, skin fat extraction or other collection strategies for obtaining a biological or chemical substance. In some embodiments, the sample is a tissue sample. For example, the tissue sample can be a fibroblast, such as a skin fibroblast.

Control values are measured from corresponding control samples from control, i.e., non-diseased, subjects. For example, in some embodiments, a MIRO1 level of a skin fibroblast from a subject is compared to a control MIRO1 level of a control skin fibroblast from a control subject.

Antibody and small molecule inhibitors may be screened using a variety of methods to detect target binding in vitro and in vivo. e.g., ELISA assays, etc. These methods include, but are not limited to, methods that measure binding affinity to a target, biodistribution of the compound within an animal or cell, or compound mediated cytotoxicity. As a first test, the antibody or small molecule may be tested for binding against the target mitochondrial transport protein. After selective binding is established, the candidate antibody may be tested for appropriate activity in an in vivo model. These and other screening methods known in the art provide information on the ability of a compound to bind to, modulate, or otherwise interact with the specified target and are a measure of the compound's efficacy.

a subjecta subjecta subjectA method to determine the MIRO1 status of a subject can comprise measuring the Miro1 response to mitochondrial depolarization using biochemical assays, western blotting or ELISA, to determine if a subject is deficient in the removal of MIRO1 following depolarization, wherein a subject deficient in the removal of MIRO1 following depolarization is selected for treatment by administration of a MIRO1 reducer. Determining the MIRO1 status can comprise detecting MIRO1 level in a subject, and comparing the MIRO1 level to a control MIRO1 level in a control subject. In some embodiments, detecting MIRO1 level comprises detecting MIRO1 in a tissue sample, such as a skin fibroblast, in the subject. In some embodiments, the method comprises an assay described in any one of the Examples.

The method can further comprise treating the subject with the MIRO1 reducer. In such instances, the MIRO1 levels can be monitored before and/or after treatment with the MIRO1 reducer. In some embodiments, the MIRO1 reducer is administered to the midbrain and/or putamen of the subject. The treatment can comprise the step of administering to the subject a therapeutically effective amount of one or more agents selected from the group consisting of levodopa, a dopamine agonist, a MAO-B inhibitor, amantadine, or an anticholinergic prior to, concurrently with, or after administering the inhibitor of a mitochondrial transport protein.

Where the MIRO1 status indicates presence of Parkinson's disease, in some embodiments, the Parkinson's disease is a PTEN-induced putative kinase 1 (PINK-1)-associated form of PD. In some cases, the Parkinson's disease is associated with a mutation in any of Parkin, leucine-rich repeat kinase 2, alpha-Synuclein, parkinson protein 7, ATPase type 13A2, phospholipase A2, group VI, DnaJ (Hsp40) homolog, subfamily C, member 6, eukaryotic translation initiation factor 4 gamma, 1, F-box protein 7, GRB10 interacting GYF protein 2, HtrA serine peptidase 2, synaptojanin 1, or vacuolar protein sorting 35 homolog. The Parkinson's disease can be a sporadic form.

The MIRO1 reducer may be administered alone or in combination with any pharmaceutically acceptable carrier or salt known in the art and as described below.

The method of detecting MIRO1 level can be by any method known in the art. In some embodiments, the method of detecting MIRO1 level comprises electrophoresis, a chromatographic method, an enzyme assay, a binding assay, or a combination thereof. In some embodiments, the method of detecting MIRO1 level comprises ELISA. In some embodiments, the method comprises an assay described in any one of the Examples.

As described herein, MIRO1 status correlates with the presence of or an increased risk of developing certain neurodegenerative disorders. In some embodiments, an elevated level of MIRO1 is associated with a neurodegenerative disorder such as Parkinson's disease. In some embodiments, the MIRO1 level in a subject is higher by about 5% or more, about 10% or more, or about 20% or more, for example, about 30% or more, about 40% or more, or about 50% or more, about 60% or more, about 70% or more, or about 80% or more, e.g. about 90% or more, about 95% or more, about 100% or more, about 200% or more, about 300% or more, about 400% or more, about 500% or more, about 600% or more, about 800% or more, about 1000% or more, about 2000% or more, about 3000% or more, relative to the control MIRO1 level in a control subject. In some embodiments, the MIRO1 level in a subject is higher by about 20% or more relative to the control MIRO1 level in a control subject. In some embodiments, the MIRO1 level in a subject is higher by about 30% or more relative to the control MIRO1 level in a control subject.

The subject may be suffering from symptoms of a neurodegenerative disorder. In one aspect, the present invention provides a method for diagnosing a subject for a neurodegenerative disorder comprising, (a) obtaining a MIRO1 level from a subject; (b) comparing the MIRO1 level to information from a control, wherein the information is predetermined to indicate absence of the neurodegenerative disorder. In some embodiments, the neurodegenerative disorder is Parkinson's disease.

Movement disorders. Movement disorders are commonly classified as those with decreased or slow movement (hypokinetic disorders) or increased movement (hyperkinetic disorders). The classic and most common hypokinetic disorder is Parkinson disease. Hyperkinetic disorders include tremor, myoclonus, dystonia, chorea, and tics.

Atypical parkinsonism refers to a group of neurodegenerative disorders other than Parkinson disease that have some features of Parkinson disease but have some different clinical features and a different pathology. As shown herein, the Miro1 status of a subject, assessed by the methods described herein, provides a distinction between PD and atypical parkonsinism. Atypical parkinsonism encompasses neurodegenerative disorders such as progressive supranuclear palsy, dementia with Lewy bodies, corticobasal ganglionic degeneration, and multiple system atrophy. Deficits that suggest neurodegenerative disorders other than Parkinson disease include gaze palsies, signs of corticospinal tract dysfunction (eg, hyperreflexia), myoclonus, autonomic dysfunction (if early or severe), cerebellar ataxia, prominent dystonia, ideomotor apraxia (inability to mimic hand motions), early dementia, early falls, and confinement to a wheelchair.

Parkinson's disease. Parkinson's disease (PD) also known as idiopathic or primary parkinsonism, hypokinetic rigid syndrome/HRS, or paralysis agitans, is a degenerative disorder of the central nervous system. The motor symptoms of Parkinson's disease result from the death of dopamine-generating neurons in the substantia nigra, a region of the midbrain, and putamen; the cause of this cell death is unknown. Early in the course of the disease, the most obvious symptoms are movement-related; these include shaking, rigidity, resting tremors, bradykinesia, postural stability, slowness of movement and difficulty with walking and gait. Later, thinking and behavioral problems may arise, with dementia, e.g. cognitive impairment, hallucinations, delusion, behavioral abnormalities, depression, disturbance of sleep and wakefulness, commonly occurring in the advanced stages of the disease, whereas depression is the most common psychiatric symptom. Other symptoms include sensory (loss of smell), sleep (disturbance of sleep and wakefulness) and emotional problems, constipation, hypotension, urinary frequency, impotence and sweating. Parkinson's disease is more common in older people, with most cases occurring after the age of 50.

Parkinson's Disease may be of the familial form or the sporadic form. By a familial form, it is meant that the disease is inherited, i.e. by the passage of a heritable gene mutation from parent to child through the gametes. A number of different heritable mutations have been associated with PD, including for example, mutations in PTEN-induced putative kinase 1 (PINK-1); Parkin (also known as RBR E3 ubiquitin protein ligase, or PARK2); leucine-rich repeat kinase 2 (LRRK2); alpha-Synuclein (SNCA, PARK4); ubiquitin carboxy-terminal hydrolase L1 (UCHL1); parkinson protein 7 (PARK7, DJ-1); ATPase type 13A2 (ATP13A2); phospholipase A2, group VI (PLA2G6); DnaJ (Hsp40) homolog, subfamily C, member 6 (DNAJC6, PARK19); eukaryotic translation initiation factor 4 gamma, 1 (EIF4G1, PARK18); F-box protein 7 (FBXO7); GRB10 interacting GYF protein 2 (GIGYF2); HtrA serine peptidase 2 (HTRA2); synaptojanin 1 (SYNJ1); or vacuolar protein sorting 35 homolog (VPS35). By a sporadic form, it is meant that the disease occurs sporadically, i.e. due to sporadic mutation of a gene, e.g. one of the aforementioned genes.

In some cases, the subject is not suffering from symptoms of a neurodegenerative disorder. In one aspect, the present invention provides a method for detecting an increased risk for a neurodegenerative disorder in a subject comprising, (a) obtaining a MIRO1 level from a subject; (b) comparing the MIRO1 level to information from a control, wherein the information is predetermined to indicate absence of the neurodegenerative disorder. In some embodiments, the neurodegenerative disorder is Parkinson's disease.

In some embodiments, a method of diagnosing a neurodegenerative disorder in a subject comprises: providing a sample from a subject suspected of having a neurodegenerative disorder; determining the level of MIRO1 in the sample; comparing the determined level of the MIRO1 to a control level of MIRO1; detecting, in the sample from the subject, an elevated level of the MIRO1 compared to the control level of MIRO1; and diagnosing the presence of a neurodegenerative disorder in the subject from the elevated level of the MIRO1. After the neurodegenerative disorder is diagnosed, the subject may be treated with a method described herein or known in the art. In some embodiments, the method comprises an assay described in any one of the Examples.

### Methods to Assay Miro1 Status

Assays are provided for determining the Miro1 status of a cell or population of cells. Miro1 is normally removed from damaged, e.g. depolarized, mitochondria to facilitate their clearance via mitophagy, but a defect in this degradation can occur, which defect can be detected using biochemical assays. The defect in degradation is strongly associated with PD or a predisposition to PD. A high percentage of Parkinson's Disease subjects are deficient in the removal of Miro1 following depolarization. However, Miro1 is efficiently degraded upon depolarization in control cells. Detection of this PD-associated defect is useful for diagnosis of PD and prognosis of susceptibility to PD, optionally including the step of treating a subject thus diagnosed; monitoring of clinical response to PD following treatment, in the context of a clinical trial, etc.; screening drugs for efficacy in reducing this defect in Miro1 degradation, etc. The methods allow assessment of Parkinson's disease accurately, early and in a clinically practical way.

The methods of analysis can be made by examining a cell composition for the presence of a Miro1 polypeptide following mitochondrial uncoupling, including chemically induced depolarization. These assay methods can be performed with cell lines, cells obtained from a subject, including without limitation biological samples such as fibroblasts, peripheral blood lymphocytes, and the like. The assay is generally performed on viable, i.e. living cells. Fibroblasts are a convenient source of cells from individuals, and can be easily obtained from subjects by a minimally-invasive, painless procedure. Cultured cells may be derived from patient or control samples; and may be modified to generate genetically-modified cells, in vitro differentiated cells, cells exposed to a candidate therapeutic agent; and the like. In some embodiments the assay is performed on a population of cells from a subject to determine the Miro1 phenotype of the individual.

In addition, cells that have been genetically altered, e.g. by transfection or transduction with recombinant genes or by antisense technology, to provide a gain or loss of genetic function, may be utilized with the invention. Methods for generating genetically modified cells are known in the art, see for example “Current Protocols in Molecular Biology”, Ausubel et al., eds, John Wiley & Sons, New York, N.Y., 2000. The genetic alteration may be a knock-out, usually where homologous recombination results in a deletion that knocks out expression of a targeted gene; or a knock-in, where a genetic sequence not normally present in the cell is stably introduced.

A variety of methods may be used in the present invention to achieve a knock-out, including site-specific recombination, expression of anti-sense or dominant negative mutations, and the like. Knockouts have a partial or complete loss of function in one or both alleles of the endogenous gene in the case of gene targeting. Preferably expression of the targeted gene product is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences are ultimately deleted from the genome, leaving a net change to the native sequence.

A cell sample may comprise, for example, at least about 10² cells, at least about 10³ cells, at least about 10⁴ cells, at least about 10⁵ cells, at least about 10⁶ cells, at least about 10⁷ cells, or more. Higher numbers of cells are optionally assayed in multiple aliquots. The cells are contacted with an agent that uncouples or depolarizes mitochondria. The concentration of agent and time of exposure is sufficient to uncouple or depolarize the mitochondria. For example, CCCP at a concentration of from about 10-100 μM, for example from about 20 to 80 μM, from about 30 to 50 μM is sufficient. Dosage of other agents may provide for activity comparable to these concentrations of CCCP. The cells are incubated for a period of time sufficient to depolarize mitochondria and initiate clearance, e.g. at least about 1 hour, at least about 2 hours, and usually not more than about 24 hours, and may be from about 1 to 24 hours, from about 2 to 20 hours, from about 3 to 18 hours, from about 4 to 14 hours, from about 5 to 10 hours, and may be from about 4 to 8 hours.

In some embodiments the agent is a mitochondria-specific uncoupler, e.g. a protonophore. Agents suitable for this purpose include FCCP, CCCP, DNP, BAM15, etc., as known in the art and described herein. Following mitochondrial depolarization, the cells are lysed and assessed for levels of Miro1, where a deficiency in Miro1 degradation relative to a control is indicative of an association with PD. The deficiency may be expressed, for example, as the ratio in Miro1 levels of a cell lysate subjected to a mitochondrial uncoupling agent relative to the same cells in the absence of an uncoupling agent. PD associated cells may have at least 2-fold the Miro1 present, at least 3-fold, at least 4-fold, or more relative to a normal cell.

A test sample from a subject is assessed for the presence of an alteration in Miro1 degradation in response to mitochondrial uncoupling. The term “alteration” in the polypeptide levels, as used herein, refers to an alteration in levels compared with a control sample. A control sample is a sample that corresponds to the test sample (e.g., is from the same type of cells), and is from a subject who is not affected by a susceptibility to PD. An alteration in the level of the polypeptide in the test sample, as compared with the control sample, is indicative of a susceptibility to PD. Protein levels can be determined by a variety of methods, including enzyme linked immunosorbent assays (ELISAs), Western blots, immunoprecipitations and immunofluorescence, etc.

For example, in one aspect, an antibody capable of binding to the polypeptide (e.g., as described above), can be used for capture or detection. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody, enzyme linked assays, radiolabeled antibodies; and the like as known in the art.

Miro1 degradation may be monitored in a variety of ways. Western blotting analysis, using an antibody that specifically binds to Miro1 can be used to identify the presence in a test sample, e.g. following cell fractionation, and may be quantitative. In other embodiments, a high throughput immunoassay is preferred. Conveniently, the removal of Miro1 is detected in a subject sample by an immunoassay, such as ELISA or other high throughput affinity assays.

In some embodiments a Miro1 assay as described above is utilized for the diagnosis and clinical monitoring of movement disorders, which diseases include, without limitation, Parkinson's disease. In some embodiments, the methods of the invention are used in determining the efficacy of a therapy for treatment of a movement disorder, e.g. in vitro, such as drug screening assays and the like; at a subject level; in the analysis of a group of subjects, e.g. in a clinical trial format; etc. Clinical trial embodiments may involve the comparison of two or more time points for a subject or group of subjects. The patient status is expected to differ between the two time points as the result of administration of a therapeutic agent, therapeutic regimen, or challenge with a disease-inducing agent to a subject undergoing treatment. The response of a subject with a movement disorder to therapy is assessed by detecting the ability of a cell sample from a subject, including without limitation a fibroblast sample, to degrade Miro1 after mitochondria damage, e.g. mitochondrial depolarization.

In some embodiments, the method comprises identifying a subject as having PD or a predisposition to PD, e.g. by criteria described above for Miro1 degradation; administering a dose of a therapeutic agent to the patient, and quantitating the degradation of Miro1 in response to mitochondrial depolarization in at least one patient sample.

In some embodiments, the methods of the invention are used in determining the efficacy of a therapy for treatment of a movement disorder, either at a subject level, or in the analysis of a group of subjects, e.g. in a clinical trial format. Such embodiments typically involve the comparison of two time points for a subject or group of subjects. The patient status is expected to differ between the two time points as the result of a therapeutic agent, therapeutic regimen, or disease challenge to a subject undergoing treatment.

For regulatory approval of treatments that offer symptomatic benefit in Parkinson's disease subjects, clinical trials have used a double-blind, placebo-controlled, parallel group design with fixed or flexible dosing strategy. A variety of efficacy outcome measures (one or more combinations of subscales of the Unified Parkinson's Disease Rating Scale [UPDRS]) and need for additional symptomatic therapy such as dopaminergic agonists, levodopa, have been used to assess the effects of treatment.

Exemplary assays for quantification of MIRO1 include those described in Hsieh C-H, Li L, Vanhauwaert R, Nguyen K T, Davis M D, Bu G, et al. Miro1 Marks Parkinson's Disease Subset and Miro1 Reducer Rescues Neuron Loss in Parkinson's Models. Cell Metab. 2019; 1131-1140; and Shaltouki A, Hsieh C-H, Kim M J, Wang X. Alpha-synuclein delays mitophagy and targeting Miro rescues neuron loss in Parkinson's models. Acta Neuropathol. 2018; 136: 607-620.

### Methods of Treatment

Provided herein is a method of treating a neurodegenerative disorder comprising administering to a subject in need thereof a Miro-reducing agent, wherein the Miro-reducing agent reduces Miro, e.g., Miro1, in Parkinson's disease cells with depolarized mitochondria to an amount equivalent to or within 5%, within 10%, within 15%, within 20%, within 25%, within 30%, within 35%, within 40%, within 45%, within 50%, within 55%, within 60%, within 65% or within 70% of the amount of Miro in control healthy cells with depolarized mitochondria not contacted with the Miro-reducing agent.

Neurodegenerative disorders included within the methods of the present invention include, but are not limited to neurological disorders that share symptoms similar to those seen in Parkinson's disease related disorders. In some cases, the neurological disorders may show symptoms similar to Parkinson's disease, atypical Parkinson's disease or Parkinson's plus disease. Examples include but are not limited to Drug-induced Parkinsonism, Progressive supranuclear Palsy, Vascular Parkinsonism, Dementia with Lewy Bodies, diffuse Lewy body disease, Corticobasal degeneration, multisystem degeneration (Shy-drager syndrome), Alzheimer's disease, Pick's disease, and progressive supranuclear palsy (Steel-Richardson syndrome). Other conditions also included within the methods of the present invention include age-related dementia and other dementias and conditions with memory loss including vascular dementia, diffuse white matter disease (Binswanger's disease), dementia of endocrine or metabolic origin, dementia of head trauma and diffuse brain damage, dementia pugilistica and frontal lobe dementia. In some cases, the neurological disorder may not respond well to dopaminergic treatments and may be caused as a result of various vascular, drug-related, infectious, toxic, structural and other known secondary causes. Drug-induced Parkinsonism may be caused by agents that block post-synaptic dopamine D2 receptors with high affinity, such as anti-psychotic and anti-emetic medications and sodium valproate, anti-depressants, reserpine, tetrabenazine etc.

A variety of subjects are suitable for treatment with an agent identified by a method of the present disclosure. Suitable subjects include any subject who displays symptoms of Parkinson's disease such as bradykinesia, repetitive movements, tremors, limb rigidity, gait and balance problems, inability to aim the eyes due to weakness of eye muscles, weakness, sensory loss, non-motor manifestations such as REM sleep behavior disorder, neuropsychiatric symptoms including mood disturbances and cognitive changes, anxiety, apathy, changes in thinking ability, level of attention or alertness and visual hallucinations, intellectual and functional deterioration, forgetfulness, personality changes, autonomic dysfunction affecting cardiovascular, respiratory, urogenital, gastrointestinal and sudomotor function, difficulties in breathing and swallowing, inability to sweat, orthostatic hypotension, pain, constipation and loss of olfaction. In some cases, the subjects may experience predominant speech or language disorder, predominant frontal presentation and gait freezing.

In certain embodiments, the subject may not display any overt symptoms of Parkinson's disease. In some cases, the subject in need may show increased susceptibility to infections, hypothermia, weaker bones, joint stiffness, arthritis, stooped posture, slowed movements, decrease in overall energy, constipation, urinary incontinence, memory loss, slower thinking, slower reflexes, difficulty with balance, decrease in visual acuity, diminished peripheral vision, hearing loss, wrinkling skin, greying hair, weight loss, loss of muscle tissue.

In some cases, the subjects may be those who have been diagnosed as having Alzheimer's disease; subjects who have suffered one or more strokes; subjects who have suffered traumatic head injury; individuals who have high serum cholesterol levels; subjects who have proteinopathies including deposits in brain tissue; subjects who have had one or more cardiac events; subjects undergoing cardiac surgery; and subjects with multiple sclerosis.

In some cases, the subject may display symptoms associated with neurological diseases that include motor neuron diseases such as amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Also other neurodegenerative disorders resulting from cerebral ischemia or infaction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).In some cases, Miro levels upon mitochondrial depolarization in cells from subjects in comparison to Miro levels upon mitochondrial depolarization in cells from control healthy subjects may be used as diagnostic assay to identify subjects that may benefit from a prophylactic use of the Miro-reducing agent to prevent aberrations in mitochondrial homeostasis. Mitochondrial homeostasis refers to a balance between the processes of mitochondrial biogenesis, mitophagy, trafficking, fission, fusion and maintenance of mitochondrial function and other processes that are at least partly dependent on Miro function.

Mitochondrial depolarization, as used here in this disclosure, refers to the process by which the potential difference across the mitochondrial membrane is reduced from its steady state level and the membrane potential of the mitochondria changes in the depolarizing direction from the resting potential, from negative to positive. The voltage gradient across the mitochondrial inner membrane is in part affected by the mitochondria permeability transition pore. In some cases, depolarization below a certain Δψm may indicate impaired mitochondrial function and can initiate mitophagy. In some cases, mitochondrial depolarization can precede the translocation of proteins such as Parkin and Pink1. In some cases, there can be a release of cytochrome accompanying mitochondrial depolarization. Mitochondrial depolarization can be monitored by rhodamine 123, Mitotracker Red, DiOC₆ or tetramethylrhodamine methyl ester (TMRE), or methyl ester (TMRM), nonylacridine orange (NAO), Saphranine O, merycyanine 540, JC-1 or JC-9 among others. Mitochondrial depolarization may be accompanied by up to 5%, up to 7%, up to 10%, up to 15%, up to 18%, up to 20%, up to 25%, up to 30%, up to 35%, up to 40%, up to 45%, up to 50%, up to 55%, up to 60%, up to 65%, up to 70%, up to 75%, up to 80%, up to 90%, up to 95% or up to 100% reduction in resting mitochondrial potential, as measured in a quantitative assay.

Mitochondrial depolarization can be induced by depolarization agent. Mitochondrial depolarization agents can be calcium dysregulation, ROS production, chemicals such as barbiturates, ginsenoside-Rh2, rotonene, complex-I inhibitors, complex-II inhibitors, complex-III inhibitors, Complex IV inhibitors, mycotoxins such as aurovertins A-E, leucinostatins A and B, venturicidin and ossamycin, efrapeptin, oligomycins A-D, vancomycin, antimycin, naturally occurring flavonoids, propranolol, local anesthetics, herbicide paraquat, pyrethroid, DDT, parathion, diethylstilbestrol, several cationic dyes and organotin compounds, uncouplers such as substituted phenols, carbonyl cyanide 4-(trifluoromethoxy) phenylhydrazone (FCCP), carbonyl cyanide meta-chlorophenylhydrazone (CCCP), trifluoromethylbenzimidazoles, salicylanilides and carbonyl cyanide phenyl hydrazones, endogenous and exogenous free fatty acids (FFA) and fatty-acid like compounds such as perfluorodecanoic acid, sulfuramide and methyl-substituted hexadecanedioic acid, peptides such as adenine nucleotide translocase, ionophores such as gramicidins (gramicidin A, D and S), nigericin, valinomycin, cationic uncouplers such as cyanine dye tri-S—C4(5), Cu2′-(o-phenanthroline)2 complex, and pentamidine, membrane active peptides such as alamethicin, Mastoparan, alternative electron acceptors such as adriamycin, paraquat, and variously substituted naphthoquinones and nitrosoamines among others.

In one aspect, the disclosure provides a method of measuring Miro degradation in cells obtained from subjectshaving a diagnosis of sporadic or familial neurodegeneration, under conditions wherein the mitochondrial membrane permeability of the cells is altered. In some cases, the cells are fibroblasts derived from tissue obtained from subjects diagnosed with sporadic or familial neurodegeneration, the method comprising obtaining tissue from the patient, preferably skin biopsy, dissecting the skin biopsy pieces into evenly sized pieces, transferring the dissected skin biopsy pieces into tissue culture plates coated with gelatin, and replacing media with complete DMEM/20% FBS media every 2-3 days until fibroblasts are confluent. In some cases, the fibroblasts are obtained from subjects diagnosed with a sporadic or familial form of Parkinson's disease. In some cases, the neurodegenerative disorder is Parkinson's disease or Parkinson's-like disease. In some cases, the subject has the disease and is asymptomatic. In some cases, the subject has a risk factor for the disease and is asymptomatic. The term sporadic Parkinson's disease, as used herein, refers to non-familial forms of Parkinson's disease. In some cases, sporadic PD is caused by environmental factors. In some cases, subjects with sporadic PD may carry genes associated with PD that have a low penetrance or an age of onset late in life which makes any familial occurrence less obvious.

In some cases, the cells are subject-specific induced pluripotent stem cells (iPSCs) related to Parkinson's disease or Parkinson's-like disease. In one embodiment the induced pluripotent stem cells are derived from human fibroblasts. In some cases, the subjectscan have a genetic form of the disease or a sporadic form of the disease. In a related embodiment the induced pluripotent stem cells are produced without the use of a retrovirus or a lentivirus. In a specific embodiment the induced pluripotent stem cells are produced with a method comprising the use of three factors, e.g., OCT4, SOX2, and KLF4. In another embodiment, the induced pluripotent stem cells are further differentiated to adopt a midbrain dopaminergic cell fate. In some cases, the induced pluripotent stem cells are differentiated to adopt the cell fate in about 20 days.

In some embodiments, the cells are neuronal cells derived from a subject having a diagnosis of Parkinson's disease or Parkinson's-like disease, the method comprising obtaining fibroblasts from the subject, dedifferentiating the fibroblasts into pluripotent stem cells, and differentiating the stem cells towards a neuronal cell fate. In one embodiment, the fibroblasts are dermal fibroblasts. In another embodiment the stem cells are differentiated towards a midbrain dopaminergic cell fate. In particular embodiments, the dedifferentiation of the fibroblasts induces pluripotency.

In some embodiments, the subject carries a genetic variation or mutation known to be associated with Parkinson's disease or Parkinson's-like disease. In other embodiments the genetic variation of interest is a copy number variation of a gene of interest. In specific embodiments, the cell line has three copies of the gene of interest. The genetic variation or mutation can be a deletion, an insertion, a complex multi-state variant, a deletion, a substitution, a transition, a transversion, or a duplication, of one or more nucleotides in the gene of interest. In exemplary embodiments, the gene of interest is selected from PARK1 (SNCA or α-synuclein), PARK2 (Parkin), PARK5 (UCHL1), PARK6 (PINK1), PARK7 (DJ-1), PARK8 (LRRK2), and PARK 11 (GIGFY2). In specific embodiments the gene of interest is PARK1 (SNCA or α-synuclein). In other specific embodiments, the gene of interest is PARK8 (LRRK2). In one embodiment, the subject carries a homozygous mutation of LRRK2 and the mutation comprises a G2019S mutation. In some embodiments, the subject has Parkinson's disease. In a specific embodiment the subject has an idiopathic form of Parkinson's disease.

In some aspects, the methods include providing cells with sequence variations or multiple copies of the gene of interest, and inducing pluripotency, multipotency or totipotency in the cells to make a cell line with the variation of the gene of interest. The methods may also include identifying a subject with a variation of the gene of interest and obtaining one or more cells from the subject. The subject-derived cells may be fibroblast cells, tumor cells, bone marrow cells, stomach cells, blood cells (such as white blood cells, blood progenitor cells), liver cells, etc. or any convenient or relevant source of cells to be obtained from the subject. The methods of generating cell lines with a copy number variation of a gene of interest may also include inducing differentiation of the cell line. The cell lines may be differentiated into any cell type of interest including endodermal, ectodermal, mesodermal, for example neural, such as neuronal cell lines, epithelial cell lines, cardiac cell lines, etc.

In some aspects, the cell lines and methods of their use include cell lines with a genetic copy number variation that is at least one duplication, for example, two or three copies of a gene. In other aspects, the copy number variation is one or more deletion, insertion, or complex multi-state variant of the gene of interest.

In some aspects, the cell lines and methods of their use include cell lines with a genetic variation that is a mutation of the gene of interest. For example, the mutation may be a deletion, substitution, transition, transversion, or duplication of one or more nucleotides in the gene of interest. In some embodiments, the mutation is a point mutation.

In one aspect, the disclosure herein provides a method of treating a neurodegenerative disorder comprising administering to a subject in need thereof a Miro-reducing agent, wherein the Miro-reducing agent reduces Miro1 in sporadic Parkinson's disease cells according to an assay comprising the following steps:
(a) plating fibroblast cells from a sporadic Parkinson's disease subject into wells of an array;
(b) 24 hours after step (a), a candidate agent is added to a first test well and the candidate agent is not added to a first control well;
(c) 10 hours after step (b), FCCP is added to a first test well and first control well;
(d) 14 hours after step (c), cells of the first test well and first control well are fixed with ice-cold 90% methanol; and
(e) the cells of the first test well and first control well are immunostained with anti-Miro1 and 4′,6-diamidino-2-phenylindole, dihydrochloride (DAPI) and imaged with a confocal microscope to measure Miro1 intensity/cell for those images of the first test well and first control well.

In some cases, the Miro-reducing agent reduces Miro1 in the first test well by up to three, up to four, up to five, up to six or up to seven standard deviations relative to the first control well. In some cases, the Miro-reducing agent reduces Miro1 in the first test well by from about 2 to about 7, or about 2 to about 5, or about 2 to 4, or about 2 to 6 standard deviations relative to the first control well. In some cases, the candidate agent reduces Miro1 in the second test well by less than one or less than a half of a standard deviation relative to the second control well, the candidate agent is a Miro-reducing agent. In some cases, Parkinson's disease cells are obtained from a familial Parkinson's disease subject. In some cases, the Parkinson's disease cells show elevated levels of Miro compared to cells from a healthy subject. In some cases, the Parkinson's disease cells shown comparable levels of Miro to a healthy subject. In some cases, the mitochondrial depolarization agent is not FCCP. In some examples, the Miro-reducing agent is a protein or protein fragment, antibody or antibody fragment, peptide, small molecule or aptamer. In some cases, the Miro is detected by ELISA or Western blotting, or fluorescence live imaging methods.

In one aspect, the disclosure provides a method of treating a neurodegenerative disorder comprising administering to a subject in need thereof a Miro-reducing agent, wherein the Miro-reducing agent reduces Miro1 in sporadic Parkinson's disease cells according to the following assay:
(a) fibroblast cells from a sporadic Parkinson's disease subject are plated into wells of an array;
(b) 24 hours after step (a), a candidate agent is added to a first test well and the candidate agent is not added to a first control well;
(c) 10 hours after step (b), FCCP is added to a first test well and first control well;
(d) 14 hours after step (c), cells of the first test well and first control well are fixed with ice-cold 90% methanol; and (e) the cells of the first test well and first control well are immunostained with anti-Miro1 and 4′,6-diamidino-2-phenylindole, dihydrochloride (DAPI) and imaged with a confocal microscope to measure Miro1 intensity/cell for those images of the first test well and first control well. In one aspect, the disclosure provides method for treatment of a neurodegenerative disorders comprising administering to a subject in need thereof a Miro-reducing agent, wherein Miro-reducing agent reduces Miro1 in sporadic Parkinson's disease cells with depolarized mitochondria by two, three, four, five or six standard deviations or more relative to sporadic Parkinson's disease cells with depolarized mitochondria not treated with the Miro-reducing agent.

In some cases, the Miro-reducing agent identified in the above assay does not reduce Miro1 in sporadic Parkinson's disease cells according to the following assay:
(a1) fibroblast cells from a sporadic Parkinson's disease subject are plated into wells of an array;
(b1) 24 hours after step (a1), the candidate agent is added to a second test well and is not added to a second control well;
(c1) 14 hours after step (b1), cells of the second test well and second control well are fixed with ice-cold 90% methanol; and
(d1) the cells of the second test well and second control well are immunostained with anti-Miro1 and 4′,6-diamidino-2-phenylindole, dihydrochloride (DAPI) and imaged with a confocal microscope to measure Miro1 intensity/cell for those images of the second test well and second control well. In one aspect, the disclosure provides method for treatment of a neurodegenerative disorder comprising administering to a subject in need thereof a Miro-reducing agent, wherein Miro-reducing agent reduces Miro1 in sporadic Parkinson's disease cells with depolarized mitochondria by two, three, four, five or six standard deviations or more relative to sporadic Parkinson's disease cells with depolarized mitochondria not treated with the Miro-reducing agent, and reduces Miro in sporadic Parkinson's disease cells by less than one standard deviation compared to sporadic Parkinson's disease cells not treated with the Miro-reducing agent.

In one aspect, the invention described herein provides a method of treating a neurodegenerative disorder comprising administering to a subject in need thereof a Miro-reducing agent, wherein the Miro-reducing agent reduces Miro1 in Parkinson's disease cells with depolarized mitochondria. In some cases, the Miro-reducing agent reduces Miro1 in Parkinson's disease cells with depolarized mitochondria, to an amount equivalent to or within 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.%, 65%, 67.5%, 70%, 72.5%, 75% of the amount of Miro in control healthy cells with depolarized mitochondria not contacted with the Miro-reducing agent. In some cases, the Miro-reducing agent reduces Miro in Parkinson's disease cells to an amount between 15%-25%, 15%-30%, 20%-30%, 20%-40%, 25%-40%, 25%-35%, 25%-45%, 30%-45%, 30%-50%, 35%-55%, 35%-60%, 35%-50%, 35%-55%, 40%-50%, 40%-55%, 40%-60%, 45%-65%, 45%-70%, 45%-75% of the amount of Miro in control healthy cells with depolarized mitochondria not contacted with the Miro-reducing agent. In some cases, the healthy cells with depolarized mitochondria are healthy cells contacted with a mitochondrial depolarizing agent.

In one aspect, the disclosure provides a method of treating a neurodegenerative disorder comprising administering to a subject in need thereof a Miro-reducing agent, wherein the Miro-reducing agent reduces Miro1 in Parkinson's disease cells with depolarized mitochondria compared to reduction of Miro1 in control depolarized sporadic Parkinson's disease cells with depolarized mitochondria not contacted with the Miro-reducing agent. In some cases, the Miro-reducing agent reduces Miro1 in Parkinson's disease cells with depolarized mitochondria by three standard deviations or more as compared to reduction of Miro1 in control depolarized sporadic Parkinson's disease cells with depolarized mitochondria not contacted with the Miro-reducing agent. In some cases, the Miro-reducing agent reduces Miro1 in Parkinson's disease cells with depolarized mitochondria by two standard deviations or more as compared to reduction of Miro1 in control depolarized sporadic Parkinson's disease cells with depolarized mitochondria not contacted with the Miro-reducing agent. In some cases, the Miro-reducing agent reduces Miro1 in Parkinson's disease cells with depolarized mitochondria by four standard deviations or more as compared to reduction of Miro1 in control depolarized sporadic Parkinson's disease cells with depolarized mitochondria not contacted with the Miro-reducing agent. In some cases, the Miro-reducing agent reduces Miro1 in Parkinson's disease cells with depolarized mitochondria by five standard deviations or more as compared to reduction of Miro1 in control depolarized sporadic Parkinson's disease cells with depolarized mitochondria not contacted with the Miro-reducing agent.

In one aspect, the disclosure provides for a method of treating a neurodegenerative disorder comprising administering to a subject in need thereof a Miro-reducing agent, wherein (a) the Miro-reducing agent reduces Miro1 in Parkinson's disease cells with depolarized mitochondria by two standard deviations or more as compared to reduction of Miro1 in control Parkinson's disease cells with depolarized mitochondria not contacted with the Miro-reducing agent; and (b) the Miro 1 reducing agent reduces Miro1 in Parkinson's disease cells with non-depolarized mitochondria by one standard deviation or less as compared to reduction of Miro1 in control Parkinson's disease cells with non-depolarized mitochondria not contacted with the Miro-reducing agent. In some cases, the Miro-reducing agent reduces Miro1 in Parkinson's disease cells with depolarized mitochondria by three standard deviations or more as compared to reduction of Miro1 in control depolarized sporadic Parkinson's disease cells with depolarized mitochondria not contacted with the Miro-reducing agent. In some cases, the Miro-reducing agent reduces Miro1 in Parkinson's disease cells with depolarized mitochondria by four standard deviations or more as compared to reduction of Miro1 in control depolarized sporadic Parkinson's disease cells with depolarized mitochondria not contacted with the Miro-reducing agent. In some cases, the Miro-reducing agent reduces Miro1 in Parkinson's disease cells with depolarized mitochondria by five standard deviations or more as compared to reduction of Miro1 in control depolarized sporadic Parkinson's disease cells with depolarized mitochondria not contacted with the Miro-reducing agent. In some cases, the Parkinson's disease cells with depolarized mitochondria are Parkinson's disease cells contacted with a mitochondrial depolarizing agent. In some cases, the Parkinson's disease cells with depolarized mitochondria are Parkinson's disease cells not contacted with a mitochondrial depolarizing agent. In some cases, the Parkinson's disease cells are sporadic Parkinson's disease cells. In some cases, the Parkinson's disease cells are familial Parkinson's disease cells. In some cases, the Parkinson's disease cells are fibroblasts. In some cases, the Parkinson's disease cells show elevated levels of Miro compared to cells from a healthy subject. In some cases, the Parkinson's disease cells shown comparable levels of Miro to a healthy subject. In some cases, the mitochondrial depolarization agent is not FCCP. In some examples, the Miro-reducing agent is a protein or protein fragment, antibody or antibody fragment, peptide, small molecule or aptamer. In some cases, the Miro is detected by ELISA or Western blotting, or fluorescence live imaging methods.

In some cases, the subject has a higher Miro level, e.g., a higher Miro1 level, than the corresponding Miro level in a control subject. For example, the subject can have about 20% or more, about 30% or more, about 40% or more, about 50% or more, about 60% or more, about 70% or more, about 80% or more, about 90% or more, about 100% or more, about 200% or more, about 300% or more, about 400% or more, about 500% or more, about 1000% or more, higher level of Miro1 than a control subject. In some cases, the subject can have a higher level of Miro1 by about 20% or more or about 30% or more compared with the Miro1 level in a control subject.

In some cases, the candidate agent is a peptide, antibody, protein, protein fragment, aptamer or small molecule. In one embodiment, the screening assays of the invention are high throughput or ultra high throughput. For example, the screening assays of the invention a may be carried out in a multi-well format, for example, a 96-well, 384-well format, or 1,536-well format, and are suitable for automation. In particular, each well of a microtiter plate can be used to run a separate assay against a selected test agent. In some cases, the concentration or incubation time effects of a single test are to be observed, every 5-10 wells can test a single test agent. It is possible to assay many plates per day; assay screens for up to about 6,000, 20,000, 50,000, or more than 100,000 different compounds using this method.

Candidate agents can be proteins or protein fragments, antibody or antibody fragment, small molecules or aptamers. The candidate agent can be in the form of a library of candidate agents, such as a combinatorial or randomized library that provides a sufficient range of diversity. Candidate agents are optionally linked to a fusion partner, e.g., targeting compounds, label or detectable moiety, rescue compounds, dimerization compounds, stabilizing compounds, addressable compounds, and other functional moieties. In some cases, the candidate agent is attached to the surface of an array. In some cases, the candidate agents are delivered in a liquid sample to the cells expressing Miro.

In some cases, the Parkinson's cells and the healthy cells express Miro protein linked to a label or reporter moiety. A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide. In some cases, levels of Miro may be detected by the use of fluorescence, luminescence, chemiluminescence, absorbance, and other optical methods. In some cases, the difference in Miro levels between the Parkinson's disease and the healthy cells is measured by computer algorithms.

In some cases, the cells may be engineered to express Miro protein tagged to enzymatic reporters that can generate a fluorescent signal or are capable of binding small molecules that can be tagged with a fluorescent moiety. In some examples, the small molecule may be tagged with a fluorescent reagent such as fluorescein, rhodamine, Texas Red, BODIPY and other commercially available molecules (such as those available from Molecular Probes/Invitrogen and other suppliers) a variety of fluorescent readouts can be generated. In other examples, ligands and other probes can be tagged directly with fluorescein or another fluorophore for detection of binding to cellular proteins; or can be tagged with enzymes such as alkaline phosphatase or horseradish peroxidase to enable indirect detection and localization of signal. In other examples, Miro may be tagged with enzymes that can be used to generate a fluorescent signal in live cells by using a specific, cell-permeable substrate that either becomes fluorescent or shifts its fluorescent spectrum upon enzymatic cleavage, resulting in shifts in fluorophore absorption or emission wavelengths. In other examples, Miro may be tagged with enzymes that can be used to generate a fluorescent signal in live cells by cleavage of a covalent assembly of emission-absorption-matched fluorophore pairs that in the covalently-assembled form sustain resonance energy transfer between the two fluorophores that is lost when the two are separated.

Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate HCS including the automated fluorescence imaging and automated microscopy systems developed by Cellomics, Amersham, TTP, Q3DM, Evotec, Universal Imaging and Zeiss. Fluorescence recovery after photobleaching (FRAP) and time lapse fluorescence microscopy have also been used to study protein mobility in living cells.

In one aspect, the invention also provides methods for identifying a diagnostic cellular phenotype comprising comparing a set of cells from a subject to cells from a subject free of Parkinson's disease wherein, the cellular phenotype detected is the degradation of Miro upon mitochondrial depolarization. In some cases, the comparison of Miro levels is performed on a computer. The cells may be fibroblasts. The cells may be induced pluripotent stem cells, or cells differentiated from induced stem cells to neural stem cells, or neurons. In one embodiment, the detected response is a change in mitochondrial function, mitochondrial fission, fusion, morphology, mitophagy, mitochondrial transport, intracellular calcium levels or other cellular features that are dependent on Miro function.

In one aspect, the invention also involves methods for determining the risk of Parkinson's disease in a subject comprising comparing at least one phenotype determined in a first set of cells derived from the subject to the at least one phenotype determined in a second set of cells derived from subjects free of Parkinson's disease and to the at least one phenotype determined in a third set of cells derived from subjects suffering from Parkinson's disease; and indicating that the subject is at high risk for Parkinson's disease if the at least one phenotype determined in the first set of cells is more similar to the at least one phenotype determined in the third set of cells than the at least one phenotype determined in the second set of cells, wherein the first, second, and third sets of cells were induced pluripotent stem cells, or were cells differentiated from induced pluripotent stem cells, and wherein the phenotype is the degradation of Miro upon mitochondrial depolarization. In one embodiment, the detected response is a change in mitochondrial function, mitochondrial fission, fusion, morphology, mitophagy, mitochondrial transport or other cellular features that are dependent on Miro function. In some cases, the comparison is performed on a computer. In some cases, the Parkinson's disease cells show elevated levels of Miro compared to cells from a healthy subject. In some cases, the Parkinson's disease cells shown comparable levels of Miro to a healthy subject. In some cases, the mitochondrial depolarization agent is not FCCP. In some examples, the Miro-reducing agent is a protein or protein fragment, antibody or antibody fragment, peptide, small molecule or aptamer. In some cases, the Miro is detected by ELISA or Western blotting, or fluorescence live imaging methods.

In one aspect, the disclosure provides for a method for selecting a subject for treatment with a therapeutic agent for a neurodegenerative disorder, comprising:
(a) collecting cells from the subject and evaluating a first control portion of the cells for the pre-depolarization Miro level in the cells;
(b) contacting a second test portion of the cells with a depolarizing agent; and
(c) evaluating the post-depolarization Miro level in the second test portion of cells contacted with the depolarizing agent and comparing the Miro level to the pre-depolarization Miro1 level in the first control portion of cells.

In some cases, the subject is treated with a therapeutic agent for neurodegenerative disorder when the post-depolarization Miro1 level in the second test portion of cells is reduced by 5% to 70%, 10% to 70%, 20% to 80%, 25% to 90%, 5% to 50%, or 10% to 50%, relative to the pre-depolarization Miro level in the first control portion of cells. In certain embodiments, the depolarizing agent reduces post-depolarization Miro level in the second test portion of cells by 5% up to 40%, up to 35%, up to 30%, up to 25%, up to 20%, up to 15%, relative to the pre-depolarization Miro level in the first control portion of cells.

In some cases, the neurodegenerative disorder is Parkinson's disease. In some cases, the cells are fibroblasts. In some cases, the cells are induced pluripotent stem cells or cells differentiated from induced pluripotent stem cells. In some cases, the cells are neurons differentiated from induced pluripotent stem cells. In some cases, the neurons are dopaminergic neurons. In some cases, the depolarization agent is FCCP.

The present invention involves methods for identifying an agent that corrects a phenotype associated with a Parkinson's disease or a predisposition for Parkinson's disease comprising contacting a first population of cells with a candidate agent, contacting a second population of cells with a control agent, assaying the two populations and identifying candidate agents as correcting the phenotype if the first population is closer to a normal phenotype following treatment than the second population, where the phenotype is cellular degradation of Miro and lowering of Miro levels following mitochondrial depolarization. In some cases, the candidate agent is a Miro-reducing agent. In some cases, the cells in both populations have been treated with a mitochondrial depolarization agent. In some cases, the cells in both populations comprise at least one endogenous allele associated with a neurodegenerative disorder or associated with a predisposition for neurodegeneration. In some cases, the Parkinson's disease cells show elevated levels of Miro compared to cells from a healthy subject. In some cases, the Parkinson's disease cells show comparable levels of Miro to a healthy subject. In some cases, the mitochondrial depolarization agent is not FCCP. In some examples, the Miro-reducing agent is a protein or protein fragment, antibody or antibody fragment, peptide, small molecule or aptamer. In some cases, the Miro is detected by ELISA or Western blotting, or fluorescence live imaging methods.

In one aspect, the disclosure provides a method of screening candidate agents to identify a Miro-reducing agent, comprising the steps of:
(a) obtaining cells deficient in Miro1 clearance;
(b) contacting a first test portion of the cells with a candidate agent and not contacting a second control portion of the cells with the candidate agent;
(c) contacting the first test portion and second control portion of cells with of step (b) with a depolarizing agent;
(d) evaluating the Miro1 levels in the first test portion relative to the second control portion.

In some cases, the candidate agent is identified as a Miro-reducing agent if the candidate agent decreases Miro levels in the first test portion by two or more, three or more, four or more, five or more, six or more, or seven or more standard deviations relative to the Miro levels in the second control portion. In some cases, the first and second test portion comprise cells obtained from a subject identified with Parkinson's disease or at risk for Parkinson's disease. In some cases, the cells are fibroblasts. In some cases, the cells are induced pluripotent stem cells or cells differentiated from pluripotent stem cells. In some cases, the cells are neurons differentiated from pluripotent stem cells. In one aspect, the disclosure provides a method of treating or preventing Parkinsonism disorders by administering to a subject an Miro-reducing agent identified by the screening methods described herein an effective amount to treat or prevent Parkinson's disease.

In one aspect, the invention provides methods for reducing the risk of drug toxicity in a huma subject of Parkinsonism, comprising contacting one or more cells generated from the subject, with a dose of a pharmacological agent, assaying the contacted one or more differentiated cells for toxicity, and prescribing or administering the pharmacological agent to the subject if, and only if, the assay is negative for toxicity in the contacted cells. The cells may be fibroblasts from the subjects, induced pluripotent cells generated from the subject, or cells differentiated from the induced pluripotent stem cell line such as neurons.

In one aspect, the disclosure provides for a method to screen cell lines with variations of a gene of interest for a candidate agent to treat a neurodegenerative disorder. The methods include contacting cells derived from a subject deficient in or suspected of being deficient in the removal of MIRO1 with a candidate agent, observing a change or lack of change in the cells, and correlating a change or lack of change with the ability of the agent to treat the disease. Such changes may be observed, for example, by staining for intracellular levels of Miro upon depolarization. The methods may further comprise comparing the cell line or its progeny with a cell line or its progeny without the variation in the gene of interest, that is, a normal cell line, or a cell line correlated to the same disorder of interest, but without the variation in the gene of interest present in the first cell line. The cells may be fibroblasts from the subjects, induced pluripotent cells generated from the subject, or cells differentiated from the induced pluripotent stem cell line such as neurons.

Methods of studying the mechanism of a neurodegenerative disorder such as Parkinson's disease are also provided herein. The methods include identifying a molecular determinant of the disorder or disease by contacting a cell line or its progeny made by the methods described herein with an agent or condition which affects a cellular pathway of interest and observing a change or lack of change in the cells. In some cases, the cellular pathway of interest is Miro degradation upon mitochondrial depolarization. In one embodiment, the cellular pathway is a change in mitochondrial function, mitochondrial fission, fusion, morphology, mitophagy, mitochondrial transport or other cellular features that are dependent on Miro function.

In some cases, the Miro-reducing agent binds to the EF hand domain of Miro. In some cases, the Miro-reducing agent increases intracellular calcium in the cell. In some cases, the Miro-reducing agent binds to the GTPase domain of Miro. In some cases, the Miro-reducing agent binds to the nucleotide binding domain of Miro. In some cases, the Miro-reducing agent binds to the microtubule binding region of Miro. In some cases, Miro-reducing agent binds to the Pink1 phosphorylation site of Miro.

In some cases, the Miro-reducing agent is a calcium channel blocker. Calcium channels are protein molecules containing pores extending through the membranes of cells or cellular organelles, which reversibly open and close, thus regulating the passage of Ca++ ions into and out of the cell or organelle. Known calcium channels include L-type, N-type, and R-type calcium channels.

In some cases, the calcium channel is an L-type channel. L-type channels are characterized by (1) “high threshold” for activation, i.e., a strong depolarization of the cell membrane in which they are located is required to open such channels; (2) large “single channel conductance”, i.e., each channel, when opened, can allow the passage of Ca+2 ions at a relatively high rate; (3) greater permeability to Ba+2 than Ca+2; and, of particular note, (4) sensitivity to high potency block by the dihydropyridine class of calcium channel antagonists such as nimodipine and nifedipine (characteristically the IC50 values for L-channel block by these drugs are below 1 μM). In most cases, the calcium “action potentials” mediated by L-type channels under normal physiological circumstances is of relatively long duration, typically no less than 100 msec. In some cases, the Miro-reducing agent is a L-type channel blocker.

In some cases, the calcium channel is an N-type channel. N-type channels are high threshold channels which are most appropriately described as dihydropyridine-insensitive but blocked by interaction with the cone snail toxin omega-conotoxin. Qualitatively, as a class, N-type channels inactivate somewhat more rapidly than L-type channels. Because there is overlap between L- and N-type channel classes in this regard, differences in inactivation kinetics do not constitute a defining characteristic. In some cases, the Miro-reducing agent is a N-type channel.

In some cases, the calcium channel is an R-type channel. R-type channels, may be characterized as high-threshold calcium channels which are relatively resistant to block by dihydropyridines and omega-conotoxin. Such channels are found in a wide variety of neurons, and are particularly abundant in cerebellar Purkinje cells. R-type channels may play a role in synaptic transmission and other processes that depend on calcium entry but are not sensitive to these blockers. In some cases, the Miro-reducing agent is a R-type channel.

In some cases, the Miro-reducing agent may be a calcium channel antagonist capable of reversibly blocking calcium channels. In some cases, the Miro-reducing agent may function by nonpermanently (i.e., noncovalently) binding to the protein molecules which constitute such channels. In some cases, the Miro-reducing agent may be a calcium channel antagonist which blocks calcium channels to a greater degree than it blocks neurotransmitter-activated channels, voltage-sensitive sodium channels and potassium channels: i.e., its IC50 for calcium channels is lower than that for such neurotransmitter-activated, sodium and potassium channels. In some cases, the Miro-reducing agent can cross the blood-brain barrier of a subject. In some cases, the Miro-reducing agent may be used to treat neurodegenerative disorders associated with excessive calcium influx into neuronal cells, including Parkinson's disease.

In some examples, the effect of a Miro-reducing agent on intracellular calcium levels may be monitored by organic synthetic fluorescent dyes such as Fura-2, Fluo-3, Fluo-4, Indo-1, Calcium green-1, Oregon green BAPTA, Rhod-2. In some examples, the effect of a Miro-reducing agent on intracellular calcium levels may be monitored by Aequorin-based luminescence calcium indicators such as Aequorin. In other examples, the effect of a Miro-reducing agent on intracellular calcium levels may be monitored by fluorescent protein based calcium indicators, such as DsRed/inverse pericam, YC 2.1, G-Camp, YC 3.1, G-Camp2, Synapcam, YC2.1, YC 2.12, Camgaroo, G-CaMP2, G-CaMP2, YC 3.12, Cer TN-L15, GCamp3 etc.

### Pharmaceutical Compositions

In some embodiments, a pharmaceutical composition comprising an effective dose of Miro1 reducer is provided, which dose may be sufficient to achieve a therapeutic level of Miro1 of at least 1 μM, at least 5 μM, at least 10 μM, at least 20 μM, up to about 1 mM, up to about 500 μM, up to about 250 μM, up to about 100 μM, up to about 75 μM, up to about 50 μM. A unit dose may be, for example, 1 μg/kg, 10 μg/kg, 100 μg/kg, 500 μg/kg, 1 mg/kg, 5 mg/kg, 10 mg/kg, 50 mg/kg, 100 mg/kg, or more.

The term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” or “vehicle” refers to a diluent, adjuvant, excipient, or vehicle with which the Miro1 reducer is administered. Such pharmaceutical carriers can be, for example, lipids, e.g. liposomes, e.g. liposome dendrimers; sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The reducer can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin, hereby incorporated by reference herein in its entirety. Such compositions will contain a therapeutically effective amount of the Miro1 reducer, preferably in purified form, together with a suitable amount of carrier so as to provide the form for proper administration to the patient. The formulation should suit the mode of administration.

The pharmaceutical composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process.

The subject pharmaceutical composition is typically sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The pharmaceutical composition may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The pharmaceutical composition comprising the lyophilized Miro1 reducer is prepared by reconstituting the lyophilized compound, for example, by using bacteriostatic Water-for-Injection.

The pharmaceutical composition can be formulated for intravenous, oral, via implant, transmucosal, transdermal, intramuscular, intrathecal, or subcutaneous administration. In some embodiments, the pharmaceutical composition is formulated for intravenous administration. In other embodiments, the pharmaceutical composition is formulated for subcutaneous administration. The following delivery systems, which employ a number of routinely used pharmaceutical carriers, are only representative of the many embodiments envisioned for administering the instant compositions.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection, an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, the pharmaceutical composition is supplied as a water free concentrate. In some embodiments, the pharmaceutical composition is supplied as a dry sterile lyophilized powder at a unit dosage of at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, or at least 75 mg.

### Methods of Administration

In the subject methods, the active agent(s) may be administered to the subject using any convenient means capable of resulting in the desired reduction in impairment of mitochondrial integrity and/or function, reduction in any associated neurological disorder, etc.

Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if de-sired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the subject.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), prefer-ably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Routes of Administration

Conventional and pharmaceutically acceptable routes of administration include intranasal, intra-muscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The com-position can be administered in a single dose or in multiple doses.

The agent can be administered to a subject using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

### Kits

Also provided are reagents, devices and kits thereof for practicing one or more of the above-described methods. The subject reagents, devices and kits thereof may vary greatly. Reagents and devices of interest include those mentioned above with respect to the methods of treating Parkinson's Disease in a subject.

Kits with unit doses of the active agent, e.g. in oral or injectable doses, are provided. In such kits, in addition to the containers containing the unit doses will be an informational package insert describing the use and attendant benefits of the drugs in treating pathological condition of interest. Preferred compounds and unit doses are those described herein above.

In one embodiment, the kit includes a MIRO1 reducer and a pharmaceutically acceptable carrier. In particular embodiments, the MIRO1 reducer and the pharmaceutically acceptable carrier are packaged separately. For example, the MIRO1 reducer may be included in the kit in a dry form, packaged in a container or vial, separate from the carrier. In other embodiments, the MIRO1 reducer is formulated in a pharmaceutically acceptable carrier.

In certain embodiments, the kit further includes at least one additional therapeutic agent. In particular embodiments, the additional therapeutic agent is selected from the group consisting of levodopa, a dopamine agonist, a MAO-B inhibitor, amantadine, an anticholinergic, a PUM1 antagonist, an SR protein antagonist, a Parkin agonist, a PINK1 agonist, an 4E-BP1 agonist, a Drp1 agonist, an Atg1 agonist, a TauS2A agonist, a Rbf1 agonist, a Dp antagonist, an E2f1 antagonist, a Polo-like kinase 2 antagonist and a Notch agonist.

In addition to the above components, the subject kits will further include instructions for practicing the subject methods of diagnosis or treatment (e.g., instructions regarding route of administration, dose, dosage regimen, site of administration, and the like). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Crossing the Blood-Brain Barrier

The blood-brain barrier limits the uptake of many therapeutic agents into the brain and spinal cord from the general circulation. Molecules which cross the blood-brain barrier use two main mechanisms: free diffusion; and facilitated transport. Because of the presence of the blood-brain barrier, attaining beneficial concentrations of a given therapeutic agent in the central nervous system (CNS) may require the use of drug delivery strategies. Delivery of therapeutic agents to the CNS can be achieved by several methods.

One method relies on neurosurgical techniques. In the case of gravely ill subjects such as accident victims or those suffering from various forms of dementia, surgical intervention is warranted despite its attendant risks. For instance, therapeutic agents can be delivered by direct physical introduction into the CNS, such as intraventricular or intrathecal injection of drugs. Intraventricular injection may be facilitated by an intraventricular catheter, for example, attached to a reservoir, such as an Ommaya reservoir. Methods of introduction may also be provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes. Neuwelt and Rappoport (1984) Fed. Proc. 43:214-219; Baba et al. (1991) J. Cereb. Blood Flow Metab. 11:638-643; and Gennuso et al. (1993) Cancer Invest. 11:638-643.

Further, it may be desirable to administer the pharmaceutical agents locally to the area in need of treatment; this may be achieved by, for example, local infusion during surgery, by injection, by means of a catheter, or by means of an implant, said implant being of a porous, non-porous, or gelatinous material, including membranes, such as silastic membranes, or fibers.

Therapeutic compounds can also be delivered by using pharmacological techniques including chemical modification or screening for an analog which will cross the blood-brain barrier. The compound may be modified to increase the hydrophobicity of the molecule, decrease net charge or molecular weight of the molecule, or modify the molecule, so that it will resemble one normally transported across the blood-brain barrier. Levin (1980) J. Med. Chem. 23:682-684; Pardridge (1991) in: Peptide Drug Delivery to the Brain; and Kostis et al. (1994) J. Clin. Pharmacol. 34:989-996.

Encapsulation of the drug in a hydrophobic environment such as liposomes is also effective in delivering drugs to the CNS. For example WO 91/04014 describes a liposomal delivery system in which the drug is encapsulated within liposomes to which molecules have been added that are normally transported across the blood-brain barrier.

Another method of formulating the drug to pass through the blood-brain barrier is to encapsulate the drug in a cyclodextrin. Any suitable cyclodextrin which passes through the blood-brain barrier may be employed, including, but not limited to, J-cyclodextrin, K-cyclodextrin and derivatives thereof. See generally, U.S. Pat. Nos. 5,017,566, 5,002,935 and 4,983,586. Such compositions may also include a glycerol derivative as described by U.S. Pat. No. 5,153,179.

Delivery may also be obtained by conjugation of a therapeutic agent to a transportable agent to yield a new chimeric transportable therapeutic agent. For example, vasoactive intestinal peptide analog (VIPa) exerted its vasoactive effects only after conjugation to a monoclonal antibody (Mab) to the specific carrier molecule transferrin receptor, which facilitated the uptake of the VI-Pa-Mab conjugate through the blood-brain barrier. Pardridge (1991); and Bickel et al. (1993) Proc. Natl. Acad Sci. USA 90:2618-2622. Several other specific transport systems have been identified, these include, but are not limited to, those for transferring insulin, or insulin-like growth factors I and II. Other suitable, non-specific carriers include, but are not limited to, pyridinium, fatty acids, inositol, cholesterol, and glucose derivatives. Certain prodrugs have been described whereby, upon entering the central nervous system, the drug is cleaved from the carrier to release the active drug. U.S. Pat. No. 5,017,566

### EXAMPLES

### Example 1: Assays To Identify Miro-Reducing Agents

### Cell Culture

Human dermal primary fibroblasts isolated from a PD patient (ND33424) and a healthy control (ND36091) were cultured in cell media DMEM (ThermoFisher, 11995-065) supplemented with 10% fetal bovine serum (Gemini Bio-Products, 900-108, heat-inactivated), 1× Anti Anti (ThermoFisher, 15240096), and 1× GlutaMax (ThermoFisher, 35050061) maintained in a 37° C., 5% CO₂ incubator with humidified atmosphere.

### Western Blot

Fibroblasts were cultured in high-glucose DMEM (SH30243.01, Invitrogen) supplemented with 10% heat-inactivated fetal bovine serum (F0926, Sigma-Aldrich, and 900-108, Gemini Bio Products) and maintained in a 37° C., 5% CO₂ incubator with humidified atmosphere. The media were refreshed every 3-4 days and split every 7-8 days. CCCP (C2759, Sigma-Aldrich) was prepared at 40 mM in DMSO fresh every time and applied at 40 μM in fresh culture medium (1:1000 dilution). IP was performed as described in (Hsieh et al., 2016).

For transfection in fibroblasts, medium was replaced with Opti-MEM (Gibco) prior to transfection. 0.5 μg of DNA or 2 μl of Lipofectamine 2000 was diluted in Opti-MEM at room temperature (22° C.) to a final volume of 50 μl in two separate tubes, and then contents of the two tubes were gently mixed, incubated for 25 min at room temperature, and subsequently added onto fibroblasts. After transfection for 6 hrs, Opti-MEM containing DNA-Lipofectamine complexes was replaced with regular culture medium. After transfection for 18 hrs, fibroblasts were live imaged, or treated with Miro1 reducer and/or CCCP.

Mitochondria were isolated from cultured human fibroblasts as described previously with minor modifications. Briefly, CCCP in DMSO or the same volume of DMSO treated fibroblasts were lifted by a cell scraper, and mechanically homogenized with a Dounce homogenizer in 750 μl isolation buffer (200 mM sucrose, 10 mM TRIS/MOPS, pH 7.4). After centrifugation at 500 g for 10 min, crude supernatant was spun at 10,000 g for 10 min to pellet intact mitochondria. Mitochondrial pellet was washed twice with isolation buffer. After this step, supernatant was referred to “cytosolic fraction (Cyto)”, and pellet was resuspended in 50 μl lysis buffer (50 mM Tris pH 8.0, 150 mM NaCl, and 1% Triton X-100-T8787, Sigma-Aldrich) with 0.25 mM phenylmethanesulfonylfluoride (P7626, Sigma-Aldrich) and protease inhibitors (Roche) referred to “mitochondrial fraction (Mito)”.

Samples were mixed 1:1 with 2×laemmli buffer (4% SDS, 20% Glycerol, 120 mM Tris-HCl, 0.02% bromophenol blue, 700 mM 2-mercaptoethanol) and boiled for 5 min prior to being loaded (Mito:Cyto=25:1) into an SDS-PAGE. 10% polyacrylamide gels (acrylamide:bis-acrylamide=29:1) and Tris-Glycine-SDS buffer (24.8 mM Tris, 192 mM glycine, 0.1% SDS) were used for electrophoresis.

After electrophoresis, nitrocellulose membranes (1620115, Bio-Rad) were used in wet transfer with Tris-Glycine buffer (24.8 mM Tris, 192 mM glycine) at 360 mA for 2 hrs on ice. Transferred membranes were first blocked overnight in phosphate-buffered saline (PBS) containing 5% fat-free milk and 0.1% tween-20 at 4° C., and then incubated with the following primary antibodies: mouse anti-Miro1 (WH0055288M1, Sigma-Aldrich) at 1:1,000, rabbit anti-Miro1 (HPA010687, Sigma-Aldrich) at 1:1,000, rabbit anti-Miro2 (HPA012624, Sigma-Aldrich) at 1:800, rabbit anti-VDAC (4661S, Cell Signaling Technology) at 1:1,000, mouse anti-Mitofusin2 (H00009927-M01, Abnova) at 1:1,000, mouse anti-Parkin (sc32282, Santa Cruz Biotechnology) at 1:500, rabbit anti-LRRK2 (NB300-268, Novus Biologicals) at 1:500, rabbit anti-OPAl (ab42364, Abcam) at 1:750, mouse anti-β-actin (A00702, Genscript) at 1:1,000, mouse anti-ubiquitin (A-104, Boston Biochem) at 1:500 or rabbit anti-GAPDH (5174S, Cell Signaling Technology) at 1:3,000, at 4° C. overnight in blocking buffer. HRP-conjugated goat anti-mouse or rabbit IgG (Jackson ImmunoResearch Laboratories) were used at 1:5-10,000. West Dura ECL Reagents (34075, GE Healthcare) were used for ECL immunoblotting. Membranes were exposed to UltraCruz autoradiography films (Santa Cruz Biotechnology) and developed on a Konica Minolta SRX-101A developer. For fluorescent Western, blots were probed with Cy5-conjugated goat anti-mouse IgG (PA45009, GE Healthcare) at 1:5,000, and scanned at 635 nm with a Molecular Dynamics Storm 860 Imager (Amersham BioSciences, Piscataway, NJ) in a linear range for fluorescent detection. Representative raw blots are in supplementary tables. Experiments were repeated for more than 3 times.

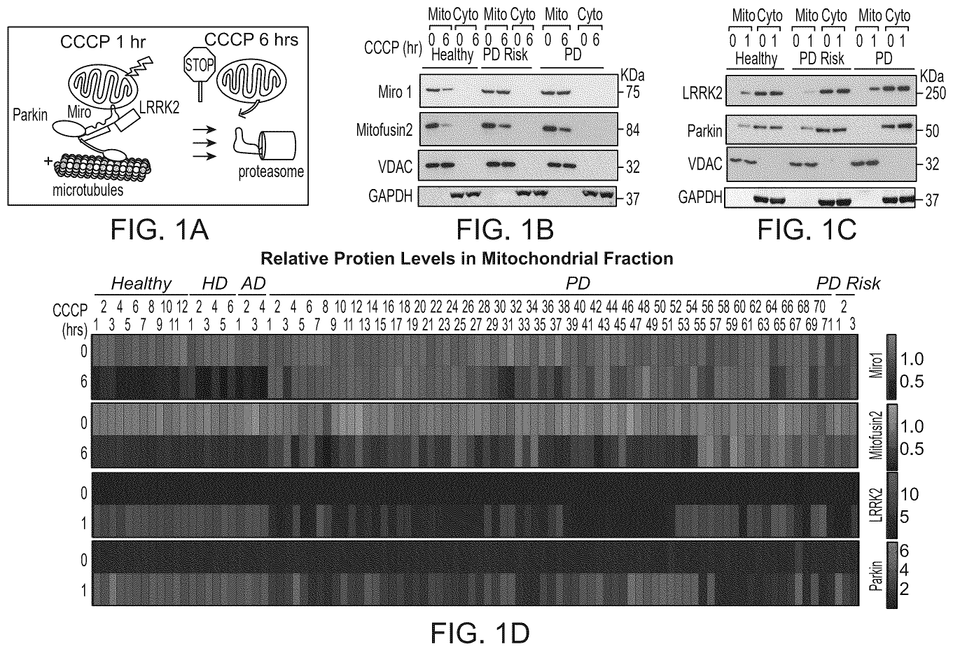

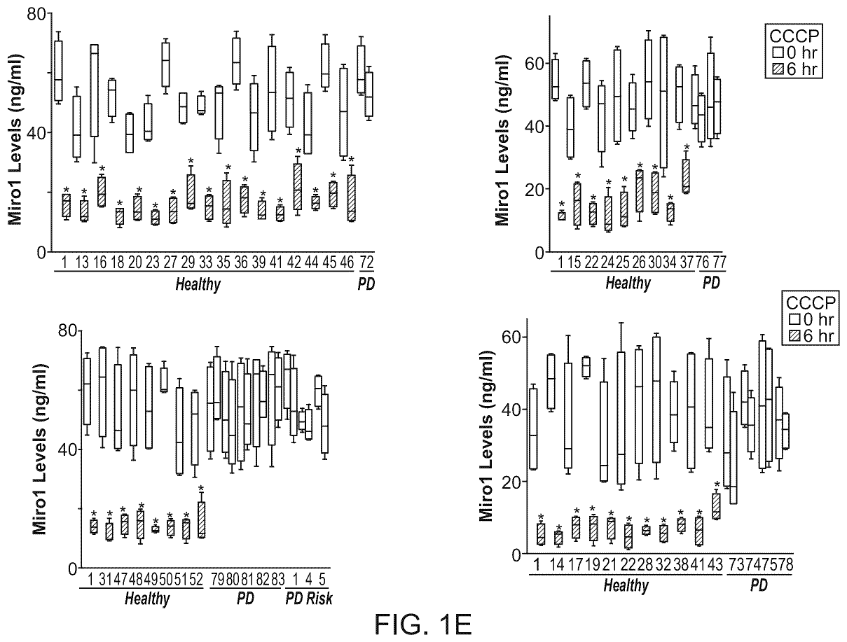

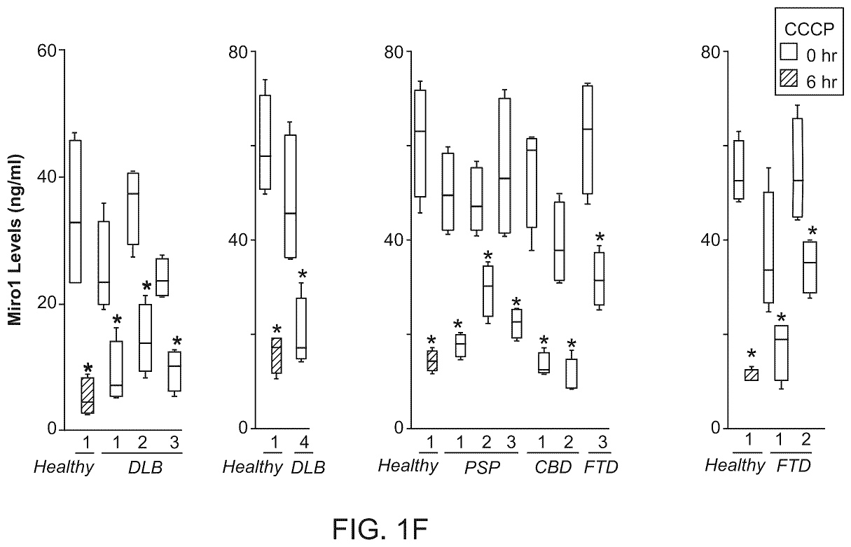

Statistics of Fibroblast Western Blotting Data. All experiments were performed in a blinded format, and the identities of the lines were un-blinded either by us (NINDS lines) or by the PPMI researchers (PPMI lines). Films were scanned or digital blots were exported as 16-bit tiff format. The intensities of protein bands were measured by ImageJ (ver. 1.48V, NIH). The intensity of each band in the mitochondrial fraction was normalized to that of the mitochondrial loading control VDAC from the same blot, and expressed as a fraction of the mean of Healthy-1 with DMSO treatment; this control was included in every independent experiment. Values of Mean±S.E.M of Miro1 were reported in Table S1A. Values of Mean of Miro1, Mitofusin2, LRRK2, and Parkin were imported into heat maps in FIG. 1D. The band intensities of VDAC were not significantly different among all fibroblast lines and conditions (p=0.8490, One-Way ANOVA Post-Hoc Tukey Test with adjustment). n=3-35 independent experiments. Mann-Whitney U Test was performed for comparing normalized Miro1 band intensities within the same subject (DMSO v.s. CCCP), and the P values were reported in Table S1A. The numbers of subjects with a P value >0.05 and <0.05 were counted respectively and used in Fisher Exact Test in Table A. Linear Regression Analysis was used to determine the correlation with the ratio of Miro1 intensity (mean intensity at CCCP/DMSO). One-Way ANOVA Post-Hoc Tukey Test with adjustment was performed for band intensities at baseline (P>0.3509 for all markers in “Cyto”+“Mito”). Statistical analyses were two-sided and performed using the Prism software (ver. 5.01, GraphPad).

### ELISA

All experiments were performed as blinded tests. 40 μM CCCP in DMSO or the same volume of DMSO alone was applied to fibroblasts for 6 hrs, and then cells were lysed in lysis buffer (100 mM Tris, 150 mM NaCl, 1 mM EGTA, 1 mM EDTA, 1% Triton X-100, 0.5% Sodium deoxycholate) with protease inhibitor cocktail (539134, Calbiochem). Cell debris was removed by centrifugation at 17,000 g for 10 min at 4° C. Microplates (MaxiSorp, NUNC) were coated with mouse anti-Miro1 (clone 4H4, WH0055288M1, Sigma-Aldrich) at 1:1,000, chicken anti-3-actin (LS-C82919, LifeSpan BioSciences) at 1:750, mouse anti-β-actin (A00702, Genscript) at 1:1000, or mouse anti-ATP5β (ab14730, Abcam) at 1:1000 in 0.1 M sodium carbonate-bicarbonate buffer (3:7, pH=9.6) overnight at room temperature with cover to avoid evaporation. After plates were washed in wash buffer (0.05% Tween 20 in PBS, pH 7.3), nonspecific binding sites were blocked in PBS with 2% BSA (BP-1600-100, Fisher scientific) for 1 hr. Next, 50 μl of cell lysate prepared from above, purified full-length Miro1 protein (0-900 ng/ml, ab163047, Abeam), or serial dilutions of cell lysates of fibroblasts (Healthy-1) or HEK cells ( 1/16×-2×) were added and incubated at room temperature for 2 hrs. After washes, plates were incubated with biotinylated rabbit anti-Miro1 (ARP44818_P050, Aviva Systems Biology) at 1:1000, or biotinylated rabbit anti-β-actin (#5057S, Cell Signaling Technology) at 1:500, in 100 μl diluent (1% BSA in PBS, pH=7.3) for 2 hrs. Next, plates were washed and incubated with horseradish peroxidase-conjugated streptavidin (21130, Thermo Scientific) at 1:2000 in 100 μl diluent for 20 min. Plates were washed again, and 100 μl of the tetramethylbenzidine liquid substrate (SB01, Life Technologies) was added and incubated for another 20 min. The colorimetric reactions were stopped by 50 μl 1 M H₂SO₄ and absorbance was read at 450 nm by a microplate reader (FlexStation 3, Molecular devices). An experiment for generating the standard curve was included in each plate and representative standard plots were shown in figures. Each data point was from 4 independent experiments with 2 technical repeats each time. Mann-Whitney U test was performed for comparing Miro1 signals within the same subject (DMSO v.s. CCCP). Basal Miro1 signals were not significantly different among all lines (P>0.1177, One-Way ANOVA Post-Hoc Tukey Test with adjustment). The distribution of data points was expressed as box-whisker plots (Extreme, Quartile, Median).

### GTPase

IPed Miro1 was eluted from protein-A beads by incubation with 60 μl 0.2 M glycine (pH 2.5) for 10 min, followed by centrifugation at 3000 g for 2 min. Supernatants (eluates) were collected and neutralized by adding an equal volume of Tris pH 8.0. Eluates were processed for colorimetric analysis of GTPase activity at room temperature by using a GTPase Activity Kit according to the manufacturer's instructions (602-0120, Novus Biologicals). 100 μl eluates were mixed with 100 μl substrate/buffer mix (20 μl 0.5 M Tris Buffer, 5 μl 0.1 M MgCl₂, 10 μl 10 mM GTP, and 65 μl ddH₂O). 200 μl of inorganic phosphate group (Pi) standard (0-50 μM) was prepared in water. Next, 50 μl PiColorLock™ mix was added into either Pi standards or samples. After 2 min, stabilizer was added and mixed thoroughly. 30 min later, absorbance was read at 650 nm by a microplate reader (FlexStation 3, Molecular devices). The assay was validated using purified Miro1 protein (ab163047, Abcam). The concentrations of purified Miro1 protein ranging from 0-900 ng/ml showed a linear dependency on the absorbances at 650 nm which reflect released Pi (R²=0.9711, P=0.0021). Omitting GTP or Miro1 eliminated the signals (Pi<3 μM). Experiments were repeated twice.

qPCR

Total RNA was extracted from at least 10⁶ cells per sample using TRIzol® (GIBCO) according to the manufacturer's instructions. Concentrations of total RNA were measured using a Nano Drop. 1 μg of total RNA was then subjected to DNA digestion using DNase I (Ambion), immediately followed by reverse transcription using the iScript Reverse Transcription Supermix (1708841, BIO-RAD). qPCR was performed using the StepOnePlus™ instrument (Thermo Fisher Scientific) and SYBR® Green Supermix (172-5270, BIO-RAD) by following the manufacturer's instructions. Human GAPDH was amplified as internal standards. Expression levels were analyzed by the StepOne™ Software (Version 2.2.2). The relative expression level of Miro1 was divided by that of GAPDH from the same experiment. Each sample was analyzed in duplicate from 4 independent biological repeats. The following primers were used:

GAPDH forward:
(SEQ ID NO: 1)
5′-ACCACAGTCCATGCCATCAC-3′
GAPDH reverse:
(SEQ ID NO: 2)
5′-TCCACCACCCTGTTGCTGT-3′
Miro1 forward:
(SEQ ID NO: 3)
5′-GGGAGGAACCTCTTCTGGA-3′
Miro1 reverse:
(SEQ ID NO: 4)
5′-ATGAAGAAAGACGTGCGGAT-3′.

### Screening Assay Protocol

Fibroblast cells were plated onto clear-bottomed/black-walled 384-well plates (EK-30091, E&K Scientific, Santa Clara, CA) at 2000 cells/well with Matrix Wellmate dispenser (Thermo Scientific, Sunnyvale, CA) and plates were incubated at 37° C., 5% CO₂ for 24 hrs. Next, chemical library compounds at defined concentrations, were added using fully automated liquid handling system Caliper Life Sciences Staccato system with a Twister II robot and a Sciclone ALH3000 (Caliper Life Sciences, Alameda, CA USA) integrated with a V&P Scientific pin tool and plates were incubated at 37° C./5% CO₂ for 10 hrs. Then, 20 μM of FCCP (Carbonyl cyanide 4-(trifluoromethoxy)phenylhydrazone, Sigma-Aldrich, C2920) in cell media was added to defined wells and plates were incubated at 37° C., 5% CO₂ for another 14 hr. Cells were fixed with ice-cold 90% methanol (Fisher, 482332) for 20 min at −20° C., incubated with blocking buffer (10% Normal Goat Serum (NGS) (ThermoFisher, 50062Z), 0.5% BSA (ThermoFisher, BP1600), 0.2% Triton X-100 (ThermoFisher, T8787) at room temperature (RT) for 15 min, and incubated with anti-Miro1 solution (Sigma-Aldrich, HPA0101687) at 1:100 in blocking buffer O/N at 4° C. Samples were washed with 1×PBS (ThermoFisher, 10010-049) using a Plate Washer multivalve (Bio-Tek, ELx405UV), incubated with goat anti-Rabbit IgG (H+L) Cross-Adsorbed, Alexa Fluor 488 (ThermoFisher, A11008) at 1:500 in blocking buffer at 25° C. for 2 hrs and washed again with 1×PBS, finally 1.0 μg/mL DAPI (ThermoFisher, D1306) in 1×PBS was added and plates were sealed using PlateLoc (Velocity 11, 01867.001). All liquids were dispensed using the Multidrop 384 (Titertek, 5840200) unless otherwise stated. Fluorescent signals in plates were automatically imaged with ImageXpress Micro (Molecular Devices, IXMicro) and data were analyzed with MetaXpress Analysis (Molecular Devices). The percent of Miro1 reduction was calculated as % Positive Cells for Miro1/Median=[(Positive Cells Miro Integrated Intensity/All Nuclear Miro Integrated Intensity)−Plate Median of this same calculation (Median % Positive Miro/Nuclear Miro in Plate Data)]/[Plate Median of this same calculation (Median % Positive Miro/Nuclear Miro in Plate Data)]. Positive cells are cells having Miro1 immunostaining. A zero value indicates no difference from the Median (Inactive). Negative value indicates a reduction in Miro. Positive value indicates an increase in Miro. The screening and data analysis were carried out together with Stanford University High-Throughput Bioscience Centers (HTBC).

### Hit Confirmation—Immunocytochemistry, Confocal Microscopy, Image Analysis

Fibroblast cells were plated in 6-well plates (VWR, 10861-554) on coverslips (Fisher scientific, 22-293232) at 400.000 cells/well and plates were incubated at 37° C., 5% CO₂ for 24 hrs. Next, newly ordered chemical compounds dissolved in DMSO were added at defined concentrations and plates were incubated at 37° C./5% CO₂ for 10 hrs. Then, FCCP (final concentration of 20 μM, Carbonyl cyanide 4-(trifluoromethoxy)phenylhydrazone, Sigma-Aldrich, C2920) in cell media was added to defined wells and plates were incubated at 37° C., 5% CO₂ for another 14 hrs. Cells were fixed with ice-cold 90% methanol (Fisher, 482332) for 20 min at −20° C., incubated with blocking buffer (10% Normal Goat Serum (NGS) (ThermoFisher, 50062Z), 0.5% BSA (ThermoFisher, BP1600), 0.2% Triton X-100 (ThermoFisher, T8787)) at 25° C. for 15 min and incubated with anti-Miro1 (Sigma-Aldrich, HPA0101687) at 1:100 in blocking buffer O/N at 4° C. Samples were washed three times with 1×PBS (ThermoFisher, 10010-049), incubated with goat anti-Rabbit IgG (H+L) Cross-Adsorbed, Alexa Fluor 488 (ThermoFisher, A11008) at 1:500 in blocking buffer at 25° C. for 2 hrs and washed again three times with 1×PBS, and finally samples were mounted with ProLong™ Glass Antifade Mountant with NucBlue™ Stain (hard-setting, Thermofisher, P36983) on glass slides and allowed to cure O/N. Samples were imaged at 25° C. with a 20×/N.A.0.60 oil Plan-Apochromat objective on a Leica SPE laser scanning confocal microscope (JH Technologies), with identical imaging parameters among different genotypes. Images were processed with ImageJ (Ver. 1.48, NIH) using the Intensity Ratio Nuclei Cytoplasm Tool (http://dev.mri.cnrs.fr/projects/imagej-macros/wiki/Intensity_Ratio_Nuclei_Cytoplasm_Tool), and average cytoplasmic intensity (the average intensity in the cytoplasm area) was plotted.

Drugs from the NIH Clinical collection library were screened at a defined 10M concentration with 4 biological repeats for each drug (377 compounds). 11 out of the 377 unique compounds were found to reduce Miro 1 protein levels by three standard deviations, following FCCP treatment, in all biological repeats. These compounds are ranked in the order of the degree of Miro1 reduction (Table 1A). 12 small molecules that reduced Miro1 protein levels in 3 out of 4 biological repeats were ranked in order of degree of Miro1 reduction. The BioMol FDA library was screened at different 5-fold doses (1.25-2.5-5-10-20 μM) and in duplicate, which identified 3 hits that partially but significantly reduced Miro1 protein levels following FCCP treatment in a dose-dependent manner. These hits were ranked in order of the degree of Miro1 reduction, with the six compounds found to reduce Miro1 protein levels by 20% or more shown (Table 1A). Moreover, an additional 12 small molecules reduced Miro1 protein levels in 3 out of 4 biological repeats (Table 1B). We next screened the Biomol FDA library with 5-fold doses (1.25-2.5-5-10-20 μM) and in duplicate. From 175 unique compounds, we identified 3 hits that partially but significantly reduced Miro1 protein levels following FCCP treatment in a dose-dependent manner. We ranked these 3 hits in the order of the degree of Miro1 reduction (Table 1A). Taken together, we identified 14 actives (2.5% primary hit rate) and 12 potential actives.

To reduce experimental bias such as the freshness the compounds, experimental reagents, and imaging devices, the 14 hits identified in the high-throughput screening were verified individually. The 14 compounds were tested at their highest screening concentrations with at least 4 biological replicates, using the same sporadic PD line. Miro1 levels were evaluated without mitochondrial depolarization (DMSO) but with the compound treatment, which reveals the drug effect on Miro1 on polarized healthy mitochondria. Samples were imaged with identical settings under the confocal microscope, and negative controls without primary antibodies yielded no signals under the imaging settings. 7 out of the initial 14 hit compounds were found to consistently reduce Miro1 protein following FCCP treatment (Table 1A). One compound, Fenbufen, appeared toxic (Table 1A). Of the 7 confirmed hits, compounds Tranilast and Benidipine HCl had no impact on basal Miro1 levels, whereas compounds Physostigmine, Pravastatin Sodium, Lofepramine, Temozolomide and Escitalopram Oxalate already reduced Miro1 protein at baseline.

### Example 2: Miro1 Marks Parkinson's Disease Subset

The identification of molecular targets and pharmacodynamic markers for Parkinson's disease (PD) will empower more effective clinical management and experimental therapies. Miro1 is localized on the mitochondrial surface and mediates mitochondrial motility. Miro1 is removed from depolarized mitochondria to facilitate their clearance via mitophagy. Here we explore the clinical utility of Miro1 for detecting PD and for gauging potential treatments. We measure the Miro1 response to mitochondrial depolarization using biochemical assays in skin fibroblasts from a broad spectrum of PD subjects, and discover that more than 94% of the subjects' fibroblast cell lines fail to remove Miro1 following depolarization.

Miro1 Is Resistant to Removal from Depolarized Mitochondria in Skin Fibroblasts from a Large Population ofPD Subjects. Skin fibroblasts can be easily obtained from subjects by a minimally-invasive, painless procedure. We aimed to determine the frequency of the Miro1 phenotype in skin fibroblasts from a large cohort of both sporadic and familial PD subjects. We fractioned mitochondria after CCCP treatment that depolarizes the mitochondrial membrane potential (Δψm). In wild-type controls at 6 hrs following treatment, both Miro1 and Mitofusin2 were removed from damaged mitochondria as detected by Western blotting (FIG. 1A, 1B).

Table SlA. Related to FIG. 1. Demographic Information and Miro1 Values. Demographic information and clinical scores are from the online databases of the consortia. Not all information is available for all subjects. Particular analyses use data from the subjects with the available information. P values are from comparing the normalized Miro1 intensities within the same subject (CCCP v.s. DMSO).

The OMM protein Mitofusin2 is a target of the PINK1-Parkin pathway, but not of LRRK2, for depolarization-triggered degradation. We included Mitofusin2 in our readout to compare its phenotypic frequency in PD with Miro1's. We screened a total of 71 PD and 3 at-risk fibroblast lines comprising the entire PD fibroblast collection at the National Institute of Neurological Disorders and Stroke (NINDS) human and cell repository and the first released PD-control cohort from the Parkinson's Progression Markers Initiative (PPMI). All subjects were diagnosed with PD and without the presence of signs for other neurological disorders. At-risk subjects are younger asymptomatic family members of the probands and harbor the same genetic mutations (in LRRK2 or SNCA). Twenty-eight subjects have a positive family history. We included 22 controls that consisted of 12 age/gender/race-matched healthy subjects recruited from the same cohorts and 10 subjects with other neurological disorders including Huntington's (HD) or Alzheimer's disease (AD) (Table A). We conducted our assay in a blinded manner.

TABLE A
Summary of the Miro1 Phenotype in All Subjects Used in This Study.

| | No. (Miro1 DMSO | No. (Miro1 DMSO | P (Fisher Exact) | No. confirmed | No. confirmed |
|---|---|---|---|---|---|
| Disease | v.s. CCCP P < 0.05) | v.s. CCCP P > 0.05) | compared to PD | by ELISA | by Western |
| PD | 5 | 78 (94%) | | 24 | 73 |
| PD Risk | 0 | 5 (100%) | 1 | 5 | 5 |
| Healthy | 52 | 0 (0%) | <0.00001 | 52 | 12 |
| HD | 6 | 0 (0%) | <0.0001 | 2 | 6 |
| AD | 4 | 0 (0%) | <0.0001 | 1 | 4 |
| DLB | 4 | 0 (0%) | <0.0001 | 4 | 0 |
| PSP | 3 | 0 (0%) | 0.0005 | 3 | 2 |
| CBD | 2 | 0 (0%) | 0.0059 | 2 | 2 |
| FTD | 3 | 0 (0%) | 0.0005 | 3 | 0 |

Fisher Exact Test was used to determine the P values compared to PD.
The Miro1 intensities with DMSO and with CCCP were compared within the same subject in either ELISA or Western by Mann-Whitney Test, and the numbers of the subjects with a P > 0.05 or <0.05 were defined as “No. (Miro1 DMSO v.s. CCCP P > 0.05 or <0.05)”.

Notably, we discovered a unifying impairment in removing Miro1 from the mitochondrial fractions at 6 hrs after CCCP treatment in 69 PD and at risk lines (93.2%). By contrast, Miro1 was efficiently removed following depolarization in every single control subject (0%) (Table A). The phenotype was more strikingly demonstrated when we imported the mean band intensities into a heat map where a lack of color change after treatment reflects the failure to remove Miro1 (FIG. 1D). The lack of color change of Miro1 occurred broadly in PD subjects. A smaller fraction of PD cell lines also failed to remove Mitofusin2 after CCCP treatment (FIG. 1D). Basal protein levels of Miro1 and Mitofusin2 were largely comparable among all lines (FIG. 1D; P>0.0906).

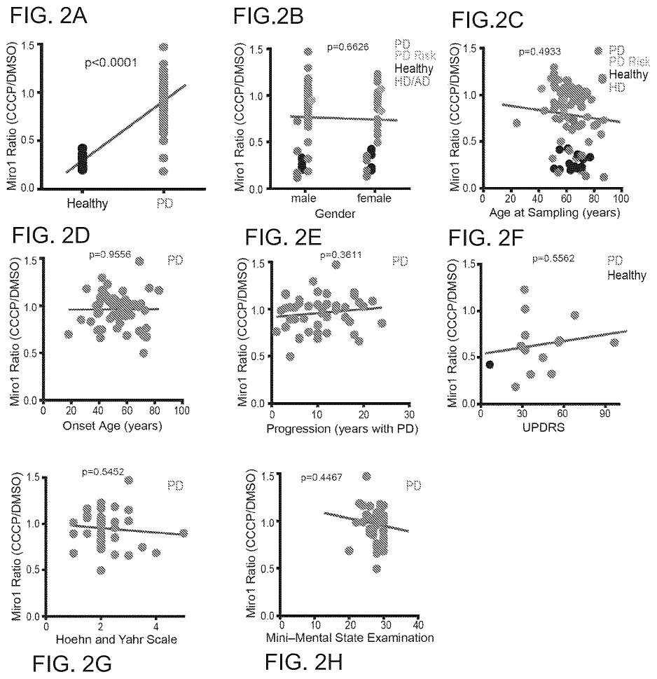

This phenotype in Miro1 removal was significantly associated with PD (P<0.00001). The ratio of Miro1 intensity (with CCCP/with DMSO) was also significantly correlated with PD (P<0.0001; FIG. 2A), but not with age (at sampling and onset) or gender (FIG. 2B-2D). There was no significant correlation between the Miro1 ratio and the disease progression (years with PD) or the clinical manifestations in subjects with the Unified Parkinson's Disease Rating Scale (UPDRS), Hoehn and Yahr Scale, or Mini-Mental Status Examination (FIG. 2E-2H). We confirmed that the cell passaging numbers within the range of 5-19 had no influence on the phenotype. Taken together, these observations show that the failure to remove Miro1 from damaged mitochondria is a common cellular defect in a large population of PD subjects.

The LRRK2 and PINK-Parkin Pathways Are Broadly Affected in PD Fibroblasts. We have previously identified two parallel molecular pathways, both of which are essential for removing Miro from the OMM of depolarized mitochondria-LRRK2 and the PINK1-Parkin axis. To investigate the mechanisms underlying Miro1 accumulation on damaged mitochondria in PD fibroblasts, we tested the hypothesis that the buildup was due to the impairments of the LRRK2 or PINK1-Parkin pathway. We have established that in wild-type control fibroblasts mitochondrial depolarization by CCCP treatment for only 1 hr triggers the recruitment of cytosolic LRRK2 and Parkin to mitochondria and Miro1, prior to Miro1 removal at 6 hrs (FIG. 1A, IC). Antibodies against LRRK2, Parkin, and Miro1 have been validated in human cells lacking the corresponding genes.

We used this readout to screen all 96 fibroblast lines, and found a variety of phenotypes in subjects' cells. Some cell lines were impaired only in recruiting LRRK2 to depolarized mitochondria, some impaired only in recruiting Parkin, and some cell lines were defective in recruiting both proteins. Because the failure to relocate LRRK2 to damaged mitochondria disrupts the following removal of Miro1 but not of Mitofusin2, this result may explain the lower frequency of the Mitofusin2 phenotype than Miro1 in those subjects (FIG. 1D). We discovered that in 7 PD cell lines the recruitment of both LRRK2 and Parkin to damaged mitochondria appeared as normal when compared to the controls, while Miro1 removal was still compromised (FIG. 1D), suggesting that additional mechanisms might be at play in those cells. Basal levels of LRRK2 and Parkin were comparable in all lines (P>0.8684). Collectively, our results provide evidence that the LRRK2 and PINK1-Parkin pathways are largely affected in PD subjects' fibroblasts, leading to a convergent downstream failure to remove Miro1 from damaged mitochondria.

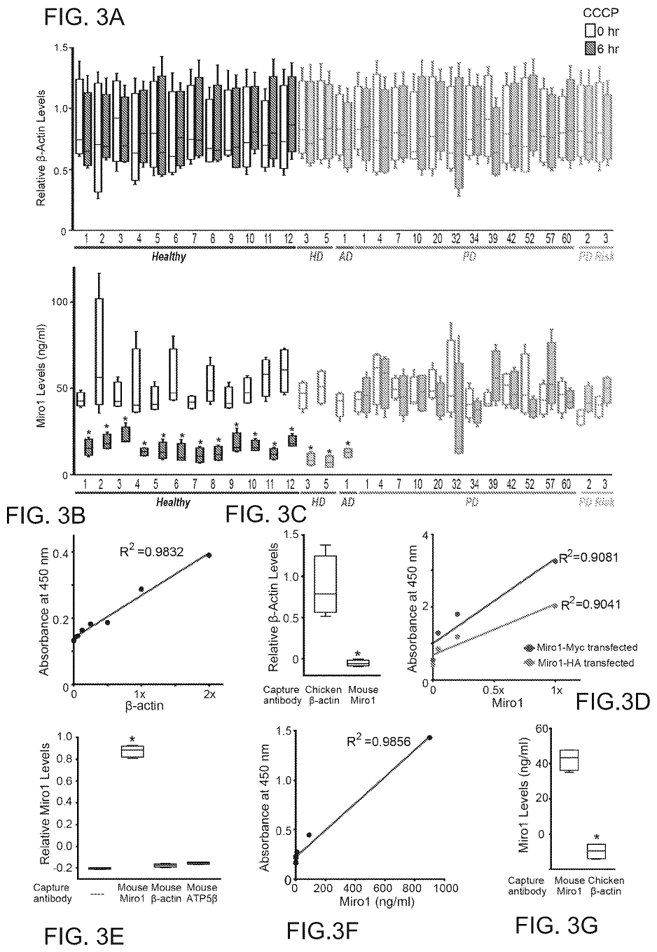

ELISA Confirms the High Frequency and Specificity of the Miro1 Phenotype for PD Fibroblasts. We established an enzyme-linked immunosorbent assay (ELISA) to detect Miro1 response to CCCP (FIG. 3) which is useful for clinical laboratory use. We examined 14 PD/risk and 15 control cell lines used in FIG. 1D. For each individual line, the result of Miro1 response to mitochondrial depolarization (FIG. 3A) was consistent with that from using mitochondrial fractionation and Western blotting (FIG. 1D, Table S1A). We used this ELISA to validate additional independent cohorts. We included 40 healthy controls and 12 PD subjects from the Stanford Alzheimer's Disease Research Center (ADRC) and the Coriell Institute (Table SIB). We discovered that Miro1 was efficiently degraded upon depolarization in all control lines, but in none of the PD subjects (FIG. 1E); this finding verified the reliability of the high frequency of Miro1 accumulation on depolarized mitochondria in the PD population (FIG. 1, Table A). We also obtained cell lines from subjects with movement disorders that exhibit clinical presentations similar to PD, including 4 of sporadic Dementia with Lewy Bodies (DLB; Stanford), 3 of Frontotemporal Degeneration (FTD; Coriell and NINDS), 2 of sporadic Corticobasal Degeneration (CBD; Mayo Clinic), and 3 of sporadic Progressive Supranuclear Palsy (PSP; Mayo Clinic) (Table SIB). Miro1 was effectively degraded after CCCP treatment in all lines (FIG. 1F), demonstrating the specificity of Miro1 accumulation for PD. The establishment of the ELISA to detect Miro1 could facilitate clinical applications of our discovery.

### Example 3: Effect of Miro-Reducing Agent on Mitochondrial Motility

Dopaminergic neurons expressing LRRK2G2019 are grown on glass coverslips and are placed in a 35-mm petridish containing the Hibernate E low-fluorescence medium (BrainBits) on a heated stage of 37° C., and are imaged with a 63×/N.A.0.9 water-immersion objective with excitation at 561 nm or 488 nm. Neurons expressing LRRK2GS2019 are split into a test pool and a control pool of neurons. The test pool is treated with a Miro-reducing agent identified in Example 1, while the control pool is not. After 10 hours, both pools of neurons are treated with FCCP for 14 hours. mitochondrial motility is measured.

Time-lapse movies are obtained continually with 3-5 sec intervals before and after Miro-reducing agent is added (10 μM) is added. Axons longer than 50 μm are selected for recording. Movies are recorded from 120 to 300 min. 250 nM TMRM is applied for 30 min when needed. For quantification, kymographs are generated from the time-lapse movies by ImageJ, representing a 100-see period either right before, or following different time points after the addition of Antimycin A. Each kymograph is then imported into a macro written in Labview (NI, TX), and individual mito-dsRed puncta are traced using a mouse-driven cursor at the center of the mito-dsRed object. Using Matlab (The MathWorks, MA), the following parameters are quantified: 1) the instantaneous velocity of each mitochondrion, 2) the average velocity of those mitochondria that are in motion, 3) the percent of time each mitochondrion is in motion, 4) stop frequency, and 5) turn back frequency. The length and intensity of mitochondria are measured using ImageJ. Neurons in the test pool with depolarized mitochondria show reduced mitochondrial motility as measured by the above parameters, whereas the control pool of neurons with depolarized mitochondria show comparable mitochondrial motility as prior to addition of Miro-reducing agent.

### Example 4: Effect of Miro-Reducing Agent on Parkinson's Disease in iPSC Neurons

LRRK2G2019S iPSC-derived neuronal cultures are treated with a mitochondrial stressor Antimycin A with a series of concentrations for 6 hrs to induce oxidative stress. LRRK2G2019S iPSC-derived neuronal cultures show an Antimycin A dose-dependent loss of neurons, depicting a vulnerability to mitochondrial stress. LRRK2G2019S neurons treated with Miro-reducing agent rescued the loss of LRRK2G2019S neurons caused by 1 or 10 μM Antimycin A, indicating a protection against neuronal loss caused by mitochondrial stress.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1A

| | | | Concentration | % Miro1 | | |
|---|---|---|---|---|---|---|
| | | Molecular | tested | decrease | Known | |
| Name | MW | formula | (μM) | in screen | target class | Description |
| Temozolomide | 194.15 | C₆H₆N₆O₂ | 10 | A | DNA | chemotherapy treatment |
| | | | | | | of anaplastic astrocytoma, |
| | | | | | | glioblastoma multiforme |
| Escitalopram | 324.39 | C₂₀H₂₁FN₂O | 10 | A | Transporter | selective serotonin |
| Oxalate | | | | | | reuptake inhibitor |
| Benidipine HCl | 505.56 | C₂₈H₃₁N₃O₆ | 10 | A | Ion-channel | calcium channel blockers |
| Glycopyrrolate | 318.43 | C₁₉H₂₈NO₃₊ | 10 | B | GPCR | long-acting muscarinic |
| | | | | | | antagonist |
| Physostigmine | 275.35 | C₁₅H₂₁N₃O₂ | 10 | B | Enzyme | acetylcholinesterase |
| | | | | | | inhibitors |
| Tranilast | 327.33 | C₁₈H₁₇NO₅ | 10 | B | Enzyme | antiasthmatics |

A = Reduction of Miro levels by >30%;
B = Reduction of Miro levels by >20% and less than 30%;
C = Reduction of Miro by >10% and less than 20%;
D = Reduction of Miro by <10%

TABLE 1B
Additional small molecules reducing Miro1 protein levels

| | | Concentration | % Miro1 | Known | |
|---|---|---|---|---|---|
| | Molecular | tested | decrease | target | |
| Name | formula | (μM) | in screen | class | Description |
| Pazufloxacin | C₁₆H₁₅FN₂O₄ | 10 | B | Unknown | antibacterials, |
| | | | | | nalidixic acid |
| | | | | | derivatives |
| Flurbiprofen | C₁₅H₁₃FO₂ | 10 | B | Enzyme | anti-inflammatory |
| | | | | | agents, ibuprofen |
| | | | | | derivatives |
| Loxoprofen | C₁₅H₁₈O₃ | 10 | B | Enzyme | anti-inflammatory |
| | | | | | agents, ibuprofen |
| | | | | | derivatives |
| Omeprazole | C₁₇H₁₉N₃O₃S | 10 | C | Transporter | antiulcer, |
| | | | | | benzimidazole |
| | | | | | derivatives |

A = Reduction of Miro levels by >30%;
B = Reduction of Miro levels by >20% and less than 30%;
C = Reduction of Miro by >10% and less than 20%;
D = Reduction of Miro by <10%

1-7. (canceled)

8. A method to determine the MIRO1 status of a subject comprising measuring the Miro1 response to mitochondrial depolarization using biochemical assays, western blotting or ELISA, to determine if a subject is deficient in the removal of MIRO1 following depolarization, wherein a subject deficient in the removal of MIRO1 following depolarization is selected for treatment by administration of a MIRO1 reducer.

9. The method of claim 8, comprising detecting MIRO1 level in the subject, and comparing the MIRO1 level to a control MIRO1 level from a control subject.

10. The method of claim 8, wherein the detecting MIRO1 level comprises ELISA.

11. The method of claim 8, wherein the detecting MIRO1 level comprises detecting MIRO1 in a sample in the subject.

12. The method of claim 11, wherein the sample comprises a skin cell.

13. The method of claim 8, wherein the MIRO1 level in the subject is higher than the control MIRO1 level from a control subject.

14. The method of claim 8, wherein the MIRO1 level in the subject is about 20% or more than the control MIRO1 level from a control subject.

15. The method of claim 8, wherein the MIRO1 level in the subject is about 30% or more than the control MIRO1 level from a control subject.

16. The method of claim 8, comprising diagnosing the subject with a neurodegenerative disorder.

17. The method of claim 16, wherein the neurodegenerative disorder is Parkinson's disease.

18. The method of claim 8, further comprising treating the subject with the MIRO1 reducer.

19. The method of claim 18, further comprising monitoring MIRO1 levels before and after treatment with the MIRO1 reducer.

20-38. (canceled)

39. A method for selecting a subject for treatment with a therapeutic agent for a neurodegenerative disorder, comprising:
(a) collecting cells from the subject and evaluating a first control portion of the cells for the pre-depolarization Miro level in the cells;
(b) contacting a second test portion of the cells with a depolarizing agent; and
(c) evaluating the post-depolarization Miro level in the second test portion of cells contacted with the depolarizing agent and comparing the Miro level to the pre-depolarization Miro level in the first control portion of cells;
wherein when the post-depolarization Miro level in the second test portion of cells is reduced by 40% or less relative to the pre-depolarization Miro level in the first control portion of cells, the subject is treated with a therapeutic agent for neurodegenerative disorder.

40. The method of claim 39, wherein when the post-depolarization Miro level in the second test portion of cells is reduced by 10% to 50% relative to the pre-depolarization Miro level in the first control portion of cells, the subject is treated with a therapeutic agent for neurodegenerative disorder.

41. The method of claim 39, wherein the neurodegenerative disorder is Parkinson's disease.

42. The method of claim 39, wherein the subject is asymptomatic for Parkinson's disease.

43. The method of claim 39, wherein the therapeutic agent is selected from levodopa and dopamine antagonists.

44. The method of claim 39, wherein the therapeutic agent is a Miro-reducing agent.

45. The method of claim 39, wherein the therapeutic agent has a molecular weight of from 100 to 2000 Daltons.

46-49. (canceled)

50. A method of improving a condition of aging, comprising administering to a subject in need thereof a Miro-reducing agent.

51-52. (canceled)


SEQUENCE LISTING (4 sequences)

SEQ ID NO: 1 - DNA, 20 bp, synthetic construct
  misc_feature 1..20: note = Synthetic construct
  source 1..20: mol_type = other DNA
accacagtccatgccatcac

SEQ ID NO: 2 - DNA, 19 bp, synthetic construct
  misc_feature 1..19: note = Synthetic construct
  source 1..19: mol_type = other DNA
tccaccaccctgttgctgt

SEQ ID NO: 3 - DNA, 19 bp, synthetic construct
  misc_feature 1..19: note = Synthetic construct
  source 1..19: mol_type = other DNA
gggaggaacctcttctgga

SEQ ID NO: 4 - DNA, 20 bp, synthetic construct
  misc_feature 1..20: note = Synthetic construct
  source 1..20: mol_type = other DNA
atgaagaaagacgtgcggat